(19) United States
(12) Patent Application Publication
(10) Pub. No.: US 2024/0065633 A1
(43) Pub. Date: Feb. 29, 2024

(54) RADAR-AIDED WARNING SYSTEM FOR PACEMAKER WEARERS AGAINST POTENTIAL ELECTROMAGNETIC INTERFERENCE BY MOBILE DEVICE

(71) Applicant: Samsung Electronics Co., Ltd., Suwon-si (KR)

(72) Inventors: Vutha Va, Plano, TX (US); Anum Ali, Plano, TX (US); Priyabrata Parida, Garland, TX (US); Saifeng Ni, Santa Clara, CA (US); Boon Loong Ng, Plano, TX (US)

(21) Appl. No.: 18/458,044

(22) Filed: Aug. 29, 2023

Related U.S. Application Data
(60) Provisional application No. 63/402,657, filed on Aug. 31, 2022.

(51) Int. Cl. A61B 5/00; G01S 13/88

(52) U.S. Cl. CPC A61B 5/686; G01S 13/886

(57) ABSTRACT

A method for warning a wearer of a pacemaker against potential electromagnetic interference by a radar-aided mobile device is provided. The method includes obtaining sensor data from a sensor of an electronic device. The method includes detecting electromagnetic interference (EMI) risk based on the sensor data obtained. The method includes determining whether a condition for executing an EMI risk mitigation function is satisfied, based on the detected EMI risk. The method includes, in response to determining that the condition for executing the EMI risk mitigation function is satisfied, performing the EMI risk mitigation function. In some embodiments, performing the EMI risk mitigation function includes at least one of: adjusting a radio frequency (RF) transmit power; and outputting an alert that warns a user of the electronic device about a pacemaker in relation to the detected EMI risk.

### CROSS-REFERENCE TO RELATED APPLICATION(S) AND CLAIM OF PRIORITY

This application claims priority under 35 U.S.C. § 119(e) to U.S. Provisional Patent Application No. 63/402,657 filed on Aug. 31, 2022. The above-identified provisional patent application is hereby incorporated by reference in its entirety.

### TECHNICAL FIELD

This disclosure relates generally to radar systems. More specifically, this disclosure relates to a radar-aided warning system for pacemaker wearers that warns against potential electromagnetic interference (EMI) caused by a mobile device.

### BACKGROUND

A pacemaker device can be subject to EMI from ambient radio frequency (RF) transmission. While typically, RF transmission by a cell phone is considered to pose a low risk for EMI, the U.S. Food and Drug Administration (FDA) recommends some precautions for pacemaker wearers to be sure that the wearer's cell phone does not cause a problem. The FDA recommends using the cell phone to the ear opposite the side of the body where the pacemaker is implanted, away from the pacemaker location, to add some extra distance between the pacemaker and the phone for better protection. Additionally, the FDA recommends avoiding keeping the phone next to the pacemaker, such as when placing the phone in a shirt or jacket pocket near the location of the heart.

### SUMMARY

This disclosure provides a radar-aided warning system for pacemaker wearers that warns against potential electromagnetic interference caused by a mobile device.

In one embodiment, a method for warning a wearer of a pacemaker against potential electromagnetic interference by a radar-aided mobile device is provided. The method includes obtaining sensor data from a sensor of an electronic device. The method includes detecting EMI risk based on the sensor data obtained. The method includes determining whether a condition for executing an EMI risk mitigation function is satisfied, based on the detected EMI risk. The method includes, in response to determining that the condition for executing the EMI risk mitigation function is satisfied, performing the EMI risk mitigation function.

In another embodiment, an electronic device for warning a wearer of a pacemaker against potential electromagnetic interference by a radar-aided mobile device is provided. The electronic device includes a transceiver and a processor operatively connected to the transceiver. The processor is configured obtain sensor data from a sensor of the electronic device. The processor is configured to detect EMI risk based on the sensor data obtained. The processor is configured to determine whether a condition for executing an EMI risk mitigation function is satisfied, based on the detected EMI risk. The processor is configured to, in response to determining that the condition for executing the EMI risk mitigation function is satisfied, perform the EMI risk mitigation function.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Definitions for other certain words and phrases may be provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an example communication system in accordance with an embodiment of this disclosure;

FIG. 2 illustrates an example electronic device in accordance with an embodiment of this disclosure;

FIG. 3 illustrates a three-dimensional view of an example electronic device that includes multiple millimeter wave (mmWave) antenna modules in accordance with an embodiment of this disclosure;

FIG. 4 illustrates an example architecture of a monostatic radar in an electronic device 400 in accordance with an embodiment of this disclosure;

FIGS. 5A-5F illustrate various scenarios in which a pacemaker wearer is a user of the electronic device that executes methods of EMI risk detection and warning, in accordance with an embodiment of this disclosure;

FIG. 6 illustrates a method of EMI risk detection and warning in accordance with an embodiment of this disclosure;

FIG. 7 illustrates an example method of direct EMI risk detection and warning that uses radar data to directly estimate the distance to the heart to determine if an EMI risk situation occurs, in accordance with an embodiment of this disclosure;

FIG. 8 illustrates an example method of estimating the distance to the heart using radar data, in accordance with an embodiment of this disclosure;

FIG. 9 illustrates an example method of estimating the distance to the heart using radar data and an external electronic device that provides a heart rate measurement, in accordance with an embodiment of this disclosure;

FIG. 10 illustrates an example method of indirect EMI risk detection and warning that uses sensor data to indirectly determine if an EMI risk situation occurs, in accordance with an embodiment of this disclosure;

FIG. 11 illustrates an example method of multiple-stage EMI risk detection and warning including a primary stage in which a primary sensor is used to indirectly determine if an EMI risk situation occurs, and a secondary stage in which the radar is subsequently activated as a secondary sensor to directly determine if the EMI risk situation occurs, in accordance with an embodiment of this disclosure;

FIG. 12 illustrates an example method of EMI risk detection and warning that includes a ternary classifier that differentiates three classes and activates radar-based direct detection of an EMI risk situation based on an “undetermined” classification from among the three classes, in accordance with an embodiment of this disclosure;

FIG. 13A illustrates an example method of direct/indirect hybrid EMI risk detection and warning that concurrently uses both radar-based direct detection of an EMI risk situation and indirect detection of the EMI risk situation based on other sensors, in accordance with an embodiment of this disclosure;

FIG. 13B illustrates a detailed view of components within block 1320 of FIG. 13A;

FIG. 14 illustrates an example method of multiple-stage EMI risk detection and warning including a primary stage in which radar-based direct detection of an EMI risk situation is performed, and a secondary stage in which a secondary sensor is subsequently used to indirectly determine if the EMI risk situation occurs, in accordance with an embodiment of this disclosure; and

FIG. 15 illustrates a method for warning a wearer of a pacemaker against potential electromagnetic interference by a radar-aided mobile device, in accordance with an embodiment of this disclosure.

### DETAILED DESCRIPTION

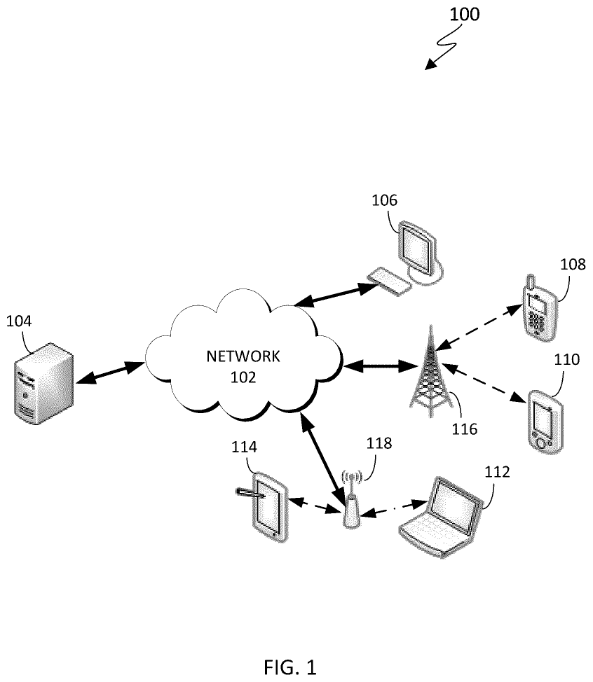

FIGS. 1 through 15, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably-arranged wireless communication device.

A pacemaker is a relatively common medical implant device. A pacemaker device can be subject to electromagnetic interference (EMI) from ambient radio frequency (RF) transmission, especially when the source of the interference is strong and in proximity to the pacemaker. EMI may cause a pacemaker to malfunction such as in one or more of the following ways: (i) the pacemaker fails to deliver the stimulating pulses that regulate the rhythm of the heart; (ii) the pacemaker delivers irregular pulses; or (iii) the pacemaker ignores the heart's own rhythm and delivers fixed rate pulses (for example, the pacemaker fails to adjust the pulse rate according to the heart's own rhythm).

This disclosure describes solutions to provide automatic warning for potential risk of EMI for pacemaker wearers to help them keep their awareness of the risk and take appropriate precautionary actions. While the FDA's current assessment suggest a low risk due to cell phones, the FDA does recommend the pacemaker wearers take simple precautionary actions to ensure additional safety. This disclosure describes systems and methods to help alert pacemaker wearers so they do not forget to take those precautionary actions at the appropriate time.

Before going into the detailed description of the embodiments, a description of what is considered an EMI risk situation is described, which description sets forth the underlying principle for embodiments of this disclosure to detect those EMI risk situations. The severity of the EMI depends on the proximity of the interferer to the pacemaker as well as the transmission power of the interferer. An EMI risk situation is where the source of interference (e.g., a smartphone) is placed at a distance to the pacemaker that is closer than a threshold distance (e.g., less than 20-30 cm) and is transmitting at a power level. In other words, a threshold distance can be defined as a function of the transmit power level. The distance threshold is smaller for a higher transmission power level. The threshold distance could be defined as a decreasing function of the transmit power level of the mobile device. An EMI risk situation occurs when the distance between the mobile device and the pacemaker is closer than a threshold distance (for the intended transmit power level). Per this definition, an EMI risk situation could be detected using the distance between the mobile device and the pacemaker.

Embodiments in accordance with this disclosure improve the accessibility settings of a mobile device, which provides extra safety and peace of mind to such patients (namely, pacemaker wearers). This disclosure describes embodiments for detecting situations (e.g., above-described situations of a cell phone in a front pocket of shirt or jacket) with potential risk of EMI or increased risk of EMI, and automatically warning the user to take precautionary action (e.g., not placing the phone in a shirt pocket) cease the situation associated with increased risk of EMI. The warning to the user of the interferer makes the user aware of the situation so that the user take precautionary action. The risk level of EMI depends on both the proximity of the pacemaker to the interferer (e.g., cell phone) as well as the signal strength of the interferer. In this disclosure, radar and/or other sensor on a mobile device is used to directly or indirectly detect EMI risk situation. The indirect EMI risk detection method uses sensors, such as IMU sensors within the mobile phone. The direct EMI risk detection method uses other sensors, such as radar within the mobile phone.

According to embodiments of this disclosure, the direct EMI risk detection method is based on radar. The pacemaker location can be approximated as the location of the heart of the person wearing the pacemaker. The location to the pacemaker can be estimated by using radar to detect the location to the heart. In this case, if the pacemaker is closer than a threshold distance to the mobile device, then the mobile device determines an occurrence of the EMI risk situation and provides a warning to the user about the detected EMI risk situation. Using a sensor like radar, such a distance could be measured directly and thus provides a direct method to detect whether an EMI risk situation is occurring. However, a radar requires line-of-sight for the detection, and in some situations (e.g., the heart is outside the field-of-view of the radar antenna or when placed in a pocket with clothing material that could cause substantial penetration loss of the radar signal), the radar might not be able to detect the heart beating. Some of those situations, such as the in-pocket placement of the smartphone, could be detected indirectly using sensor information from other sensors.

While the radar itself also emits RF energy, the radar emission is less problematic than RF emissions association with communications, especially when using a high frequency radar, such as a millimeter wave (mmWave) radar (e.g., at 60 GHz). There are two reasons for this. First, the radar uses a much smaller duty cycle than a communication transmission because radar pulses can be very short. Second, at high frequency such as mmWave, the RF energy emitted from the radar does not penetrate deeply into the human body. Most of the RF energy emitted from the radar that is incident upon the human body is absorbed, and does not penetrate deeper than approximately 1 millimeter under the skin. RF energy emitted from the radar is likely to be negligible energy if any could reach a pacemaker.

According to embodiments of this disclosure, the indirect EMI risk detection method is based on using other sensors (such as an Inertial Measurement Unit (IMU)) and/or radar. Sensor data is used to detect if the mobile device is placed in a shirt pocket or jacket pocket. If it is detected that the mobile device is in a shirt pocket or a jacket pocket, then the mobile device determines an occurrence of the EMI risk situation, and the mobile device alerts the user about the detected EMI risk situation. According to some embodiments of this disclosure, the direct and indirect EMI risk detection methods are used together to improve the accuracy and reliability of the detection.

FIG. 1 illustrates an example communication system in accordance with an embodiment of this disclosure. The embodiment of the communication system 100 shown in FIG. 1 is for illustration only. Other embodiments of the communication system 100 can be used without departing from the scope of this disclosure.

The communication system 100 includes a network 102 that facilitates communication between various components in the communication system 100. For example, the network 102 can communicate IP packets, frame relay frames, Asynchronous Transfer Mode (ATM) cells, or other information between network addresses. The network 102 includes one or more local area networks (LANs), metropolitan area networks (MANs), wide area networks (WANs), all or a portion of a global network such as the Internet, or any other communication system or systems at one or more locations.

In this example, the network 102 facilitates communications between a server 104 and various client devices 106-114. The client devices 106-114 may be, for example, a smartphone, a tablet computer, a laptop, a personal computer, a wearable device, a head mounted display, or the like. The server 104 can represent one or more servers. Each server 104 includes any suitable computing or processing device that can provide computing services for one or more client devices, such as the client devices 106-114. Each server 104 could, for example, include one or more processing devices, one or more memories storing instructions and data, and one or more network interfaces facilitating communication over the network 102.

Each of the client devices 106-114 represent any suitable computing or processing device that interacts with at least one server (such as the server 104) or other computing device(s) over the network 102. The client devices 106-114 include a desktop computer 106, a mobile telephone or mobile device 108 (such as a smartphone), a PDA 110, a laptop computer 112, and a tablet computer 114. However, any other or additional client devices could be used in the communication system 100. Smartphones represent a class of mobile devices 108 that are handheld devices with mobile operating systems and integrated mobile broadband cellular network connections for voice, short message service (SMS), and Internet data communications. In certain embodiments, any of the client devices 106-114 can emit and collect radar signals via a radar transceiver. In certain embodiments, the client devices 106-114 are able to sense the presence of an object located close to the client device.

In this example, some client devices 108 and 110-114 communicate indirectly with the network 102. For example, the mobile device 108 and PDA 110 communicate via one or more base stations 116, such as cellular base stations or eNodeBs (eNBs) or gNodeBs (gNBs). Also, the laptop computer 112 and the tablet computer 114 communicate via one or more wireless access points 118, such as IEEE 802.11 wireless access points. Note that these are for illustration only and that each of the client devices 106-114 could communicate directly with the network 102 or indirectly with the network 102 via any suitable intermediate device(s) or network(s). In certain embodiments, any of the client devices 106-114 transmit information securely and efficiently to another device, such as, for example, the server 104.

Although FIG. 1 illustrates one example of a communication system 100, various changes can be made to FIG. 1. For example, the communication system 100 could include any number of each component in any suitable arrangement. In general, computing and communication systems come in a wide variety of configurations, and FIG. 1 does not limit the scope of this disclosure to any particular configuration. While FIG. 1 illustrates one operational environment in which various features disclosed in this patent document can be used, these features could be used in any other suitable system.

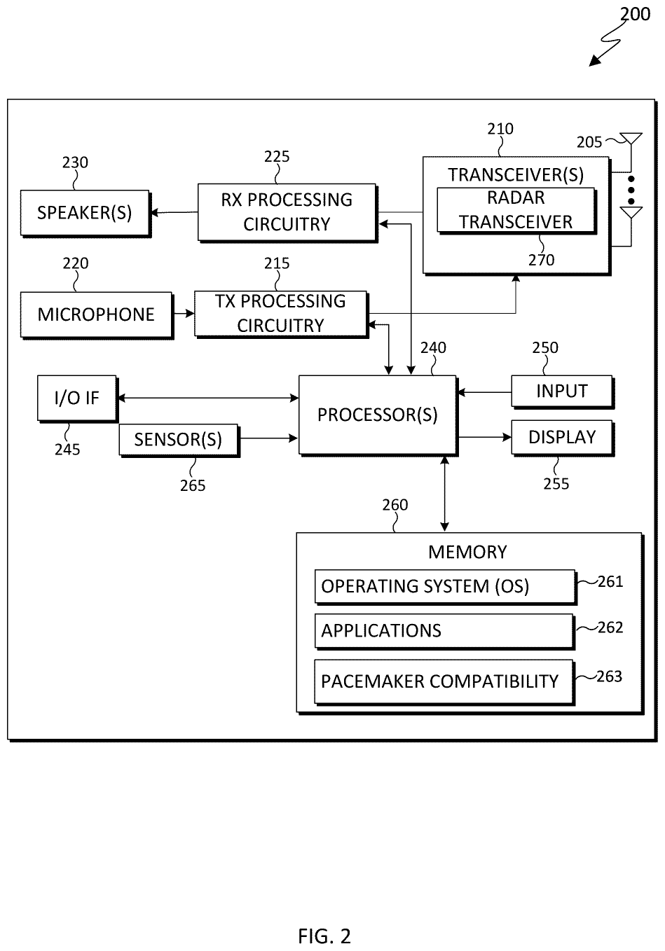

FIG. 2 illustrates an example electronic device in accordance with an embodiment of this disclosure. In particular, FIG. 2 illustrates an example electronic device 200, and the electronic device 200 could represent the server 104 or one or more of the client devices 106-114 in FIG. 1. The electronic device 200 can be a mobile communication device, such as, for example, a mobile station, a subscriber station, a wireless terminal, a desktop computer (similar to the desktop computer 106 of FIG. 1), a portable electronic device (similar to the mobile device 108, the PDA 110, the laptop computer 112, or the tablet computer 114 of FIG. 1), a robot, and the like.

As shown in FIG. 2, the electronic device 200 includes transceiver(s) 210, transmit (TX) processing circuitry 215, a microphone 220, and receive (RX) processing circuitry 225. The transceiver(s) 210 can include, for example, a RF transceiver, a BLUETOOTH transceiver, a WiFi transceiver, a ZIGBEE transceiver, an infrared transceiver, and various other wireless communication signals. The electronic device 200 also includes a speaker 230, a processor 240, an input/output (I/O) interface (IF) 245, an input 250, a display 255, a memory 260, and a sensor 265. The memory 260 includes an operating system (OS) 261, and one or more applications 262.

The transceiver(s) 210 can include an antenna array 205 including numerous antennas. The antennas of the antenna array can include a radiating element composed of a conductive material or a conductive pattern formed in or on a substrate. The transceiver(s) 210 transmit and receive a signal or power to or from the electronic device 200. The transceiver(s) 210 receives an incoming signal transmitted from an access point (such as a base station, WiFi router, or BLUETOOTH device) or other device of the network 102 (such as a WiFi, BLUETOOTH, cellular, 5G, 6G, LTE, LTE-A, WiMAX, or any other type of wireless network). The transceiver(s) 210 down-converts the incoming RF signal to generate an intermediate frequency or baseband signal. The intermediate frequency or baseband signal is sent to the RX processing circuitry 225 that generates a processed baseband signal by filtering, decoding, and/or digitizing the baseband or intermediate frequency signal. The RX processing circuitry 225 transmits the processed baseband signal to the speaker 230 (such as for voice data) or to the processor 240 for further processing (such as for web browsing data).

The TX processing circuitry 215 receives analog or digital voice data from the microphone 220 or other outgoing baseband data from the processor 240. The outgoing baseband data can include web data, e-mail, or interactive video game data. The TX processing circuitry 215 encodes, multiplexes, and/or digitizes the outgoing baseband data to generate a processed baseband or intermediate frequency signal. The transceiver(s) 210 receives the outgoing processed baseband or intermediate frequency signal from the TX processing circuitry 215 and up-converts the baseband or intermediate frequency signal to a signal that is transmitted.

The processor 240 can include one or more processors or other processing devices. The processor 240 can execute instructions that are stored in the memory 260, such as the OS 261 in order to control the overall operation of the electronic device 200. For example, the processor 240 could control the reception of downlink (DL) channel signals and the transmission of uplink (UL) channel signals by the transceiver(s) 210, the RX processing circuitry 225, and the TX processing circuitry 215 in accordance with well-known principles. The processor 240 can include any suitable number(s) and type(s) of processors or other devices in any suitable arrangement. For example, in certain embodiments, the processor 240 includes at least one microprocessor or microcontroller. Example types of processor 240 include microprocessors, microcontrollers, digital signal processors, field programmable gate arrays, application specific integrated circuits, and discrete circuitry. In certain embodiments, the processor 240 can include a neural network.

The processor 240 is also capable of executing other processes and programs resident in the memory 260, such as operations that receive and store data. The processor 240 can move data into or out of the memory 260 as required by an executing process. In certain embodiments, the processor 240 is configured to execute the one or more applications 262 based on the OS 261 or in response to signals received from external source(s) or an operator. Example, applications 262 can include a multimedia player (such as a music player or a video player), a phone calling application, a virtual personal assistant, and the like. In certain embodiments, the processor 240 is configured to execute a pacemaker compatibility module 263 that configures (e.g., activate or deactivate) a pacemaker compatibility mode. The pacemaker compatibility module 263 facilitates user selection to activate or to deactivate the pacemaker compatibility mode. In some embodiments, the pacemaker compatibility module 263 improves the accessibility settings associated with the OS 261 of the electronic device 200 by providing a user interface for receiving a user selection of one state selected from an activated state and a deactivated state of the pacemaker compatibility mode. Also, the user selection can indicate a level of concern (aversion) regarding EMI to the pacemaker, such as a selection of one concern level from among a high level or nominal level of concern.

The processor 240 is also coupled to the I/O interface 245 that provides the electronic device 200 with the ability to connect to other devices, such as client devices 106-114. The I/O interface 245 is the communication path between these accessories and the processor 240.

The processor 240 is also coupled to the input 250 and the display 255. The operator of the electronic device 200 can use the input 250 to enter data or inputs into the electronic device 200. The input 250 can be a keyboard, touchscreen, mouse, track ball, voice input, or other device capable of acting as a user interface to allow a user in interact with the electronic device 200. For example, the input 250 can include voice recognition processing, thereby allowing a user to input a voice command. In another example, the input 250 can include a touch panel, a (digital) pen sensor, a key, or an ultrasonic input device. The touch panel can recognize, for example, a touch input in at least one scheme, such as a capacitive scheme, a pressure sensitive scheme, an infrared scheme, or an ultrasonic scheme. The input 250 can be associated with the sensor(s) 265, a camera, and the like, which provide additional inputs to the processor 240. The input 250 can also include a control circuit. In the capacitive scheme, the input 250 can recognize touch or proximity.

The display 255 can be a liquid crystal display (LCD), light-emitting diode (LED) display, organic LED (OLED), active-matrix OLED (AMOLED), or other display capable of rendering text and/or graphics, such as from websites, videos, games, images, and the like. The display 255 can be a singular display screen or multiple display screens capable of creating a stereoscopic display. In certain embodiments, the display 255 is a heads-up display (HUD).

The memory 260 is coupled to the processor 240. Part of the memory 260 could include a RAM, and another part of the memory 260 could include a Flash memory or other ROM.

The memory 260 can include persistent storage (not shown) that represents any structure(s) capable of storing and facilitating retrieval of information (such as data, program code, and/or other suitable information). The memory 260 can contain one or more components or devices supporting longer-term storage of data, such as a read only memory, hard drive, Flash memory, or optical disc.

The electronic device 200 further includes one or more sensors 265 that can meter a physical quantity or detect an activation state of the electronic device 200 and convert metered or detected information into an electrical signal. For example, the sensor 265 can include one or more buttons for touch input, a camera, a gesture sensor, optical sensors, cameras, one or more inertial measurement units (IMUs), such as a gyroscope or gyro sensor, and an accelerometer. The sensor 265 can also include an air pressure sensor, a magnetic sensor or magnetometer, a grip sensor, a proximity sensor, an ambient light sensor, a bio-physical sensor, a temperature/humidity sensor, an illumination sensor, an Ultraviolet (UV) sensor, an Electromyography (EMG) sensor, an Electroencephalogram (EEG) sensor, an Electrocardiogram (ECG) sensor, an IR sensor, an ultrasound sensor, an iris sensor, a fingerprint sensor, a color sensor (such as a Red Green Blue (RGB) sensor), and the like. The sensor 265 can further include control circuits for controlling any of the sensors included therein. Any of these sensor(s) 265 may be located within the electronic device 200 or within a secondary device operably connected to the electronic device 200.

The electronic device 200 as used herein can include a transceiver that can both transmit and receive radar signals. For example, the transceiver(s) 210 includes a radar transceiver 270, as described more particularly below. In this embodiment, one or more transceivers in the transceiver(s) 210 is a radar transceiver 270 that is configured to transmit and receive signals for detecting and ranging purposes. For example, the radar transceiver 270 may be any type of transceiver including, but not limited to a WiFi transceiver, for example, an 802.11ay transceiver. The radar transceiver 270 can operate both radar and communication signals concurrently. The radar transceiver 270 includes one or more antenna arrays, or antenna pairs, that each includes a transmitter (or transmitter antenna) and a receiver (or receiver antenna). The radar transceiver 270 can transmit signals at a various frequencies. For example, the radar transceiver 270 can transmit signals at frequencies including, but not limited to, 6 GHz, 7 GHz, 8 GHz, 28 GHz, 39 GHz, 60 GHz, and 77 GHz. In some embodiments, the signals transmitted by the radar transceiver 270 can include, but are not limited to, millimeter wave (mmWave) signals. The radar transceiver 270 can receive the signals, which were originally transmitted from the radar transceiver 270, after the signals have bounced or reflected off of target objects in the surrounding environment of the electronic device 200. In some embodiments, the radar transceiver 270 can be associated with the input 250 to provide additional inputs to the processor 240.

In certain embodiments, the radar transceiver 270 is a monostatic radar. A monostatic radar includes a transmitter of a radar signal and a receiver, which receives a delayed echo of the radar signal, which are positioned at the same or similar location. For example, the transmitter and the receiver can use the same antenna or nearly co-located while using separate, but adjacent antennas. Monostatic radars are assumed coherent such that the transmitter and receiver are synchronized via a common time reference. FIG. 4, below, illustrates an example monostatic radar.

In certain embodiments, the radar transceiver 270 can include a transmitter and a receiver. In the radar transceiver 270, the transmitter can transmit millimeter wave (mmWave) signals. In the radar transceiver 270, the receiver can receive the mmWave signals originally transmitted from the transmitter after the mmWave signals have bounced or reflected off of target objects in the surrounding environment of the electronic device 200. The processor 240 can analyze the time difference between when the mmWave signals are transmitted and received to measure the distance of the target objects from the electronic device 200. Based on the time differences, the processor 240 can generate an image of the object by mapping the various distances.

Although FIG. 2 illustrates one example of electronic device 200, various changes can be made to FIG. 2. For example, various components in FIG. 2 can be combined, further subdivided, or omitted and additional components can be added according to particular needs. As a particular example, the processor 240 can be divided into multiple processors, such as one or more central processing units (CPUs), one or more graphics processing units (GPUs), one or more neural networks, and the like. Also, while FIG. 2 illustrates the electronic device 200 configured as a mobile telephone, tablet, or smartphone, the electronic device 200 can be configured to operate as other types of mobile or stationary devices.

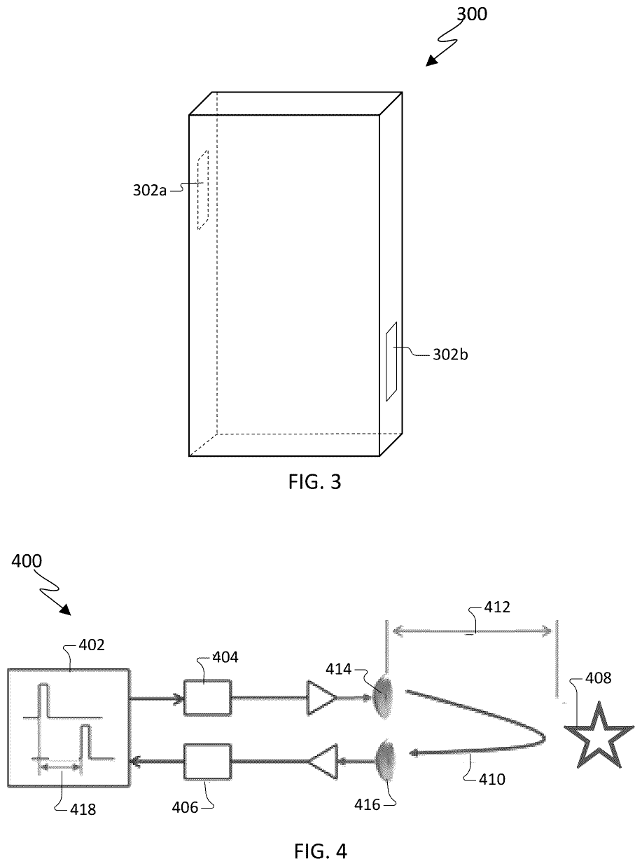

FIG. 3 illustrates a three-dimensional view of an example electronic device 300 that includes multiple millimeter wave (mmWave) antenna modules 302 in accordance with an embodiment of this disclosure. The electronic device 300 could represent one or more of the client devices 106-114 in FIG. 1 or the electronic device 200 in FIG. 2. The embodiments of the electronic device 300 illustrated in FIG. 3 are for illustration only, and other embodiments can be used without departing from the scope of the present disclosure.

As used herein, the term “module” may include a unit implemented in hardware, software, or firmware, and may interchangeably be used with other terms, for example, “logic,” “logic block,” “part,” or “circuitry.” A module may be a single integral component, or a minimum unit or part thereof, adapted to perform one or more functions. For example, according to an embodiment, the module may be implemented in a form of an application-specific integrated circuit (ASIC).

The first antenna module 302a and the second antenna module 302b are positioned at the left and the right edges of the electronic device 300. For simplicity, the first and second antenna modules 302a-302b are generally referred to as an antenna module 302. In certain embodiments, the antenna module 302 includes an antenna panel, circuitry that connects the antenna panel to a processor (such as the processor 240 of FIG. 2), and the processor.

The electronic device 300 can be equipped with multiple antenna elements. For example, the first and second antenna modules 302a-302b are disposed in the electronic device 300 where each antenna module 302 includes one or more antenna elements. The electronic device 300 uses the antenna module 302 to perform beamforming when the electronic device 300 attempts to establish a connection with a base station (for example, base station 116).

FIG. 4 illustrates an example architecture of a monostatic radar in an electronic device 400 in accordance with an embodiment of this disclosure. The embodiments of the architecture of the monostatic radar illustrated in FIG. 4 are for illustration only and other embodiments can be used without departing from the scope of the present disclosure.

The electronic device 400 that includes a processor 402, a transmitter 404, and a receiver 406. The electronic device 400 can be similar to any of the client devices 106-114 of FIG. 1, the electronic device 200 of FIG. 2, or the electronic device 300 of FIG. 3. The processor 402 is similar to the processor 240 of FIG. 2. Additionally, the transmitter 404 and the receiver 406 can be included within the radar transceiver 270 of FIG. 2. The radar can be used to detect the range, velocity and/or angle of a target object 408. Operating at mmWave frequency with GHz of bandwidth (e.g., 2, 3, 5 or 7 GHz bandwidth), the radar can be useful for applications such as proximity sensing, gesture recognition, liveness detection, mmWave blockage detection, and so on.

The transmitter 404 transmits a signal 410 (for example, a monostatic radar signal) to the target object 408. The target object 408 is located a distance 412 from the electronic device 400. In certain embodiments, the target object 408 corresponds to the objects that form the physical environment around the electronic device 400. For example, the transmitter 404 transmits a signal 410 via a transmit antenna 414. The signal 410 reflects off of the target object 408 and is received by the receiver 406 as a delayed echo, via a receive antenna 416. The signal 410 represents one or many signals that can be transmitted from the transmitter 404 and reflected off of the target object 408. The processor 402 can identify the information associated with the target object 408 based on the receiver 406 receiving the multiple reflections of the signals.

The processor 402 analyzes a time difference 418 from when the signal 410 is transmitted by the transmitter 404 and received by the receiver 406. The time difference 418 is also referred to as a delay, which indicates a delay between the transmitter 404 transmitting the signal 410 and the receiver 406 receiving the signal after the signal is reflected or bounced off of the target object 408. Based on the time difference 418, the processor 402 derives the distance 412 between the electronic device 400, and the target object 408. The distance 412 can change when the target object 408 moves while electronic device 400 is stationary. The distance 412 can change when the electronic device 400 moves while the target object 408 is stationary. Also, the distance 412 can change when the electronic device 400 and the target object 408 are both moving. As described herein, the electronic device 400 that includes the architecture of a monostatic radar is also referred to as a radar 400.

The signal 410 can be a radar pulse as a realization of a desired “radar waveform,” modulated onto a radio carrier frequency. The transmitter 404 transmits the radar pulse signal 410 through a power amplifier and transmit antenna 414, either omni-directionally or focused into a particular direction. A target (such as target 408), at a distance 412 from the location of the radar (e.g., location of the transmit antenna 414) and within the field-of-view of the transmitted signal 410, will be illuminated by RF power density pₜ (in units of W/m2) for the duration of the transmission of the radar pulse. Herein, the distance 412 from the location of the radar to the location of the target 408 is simply referred to as “R” or as the “target distance.” To first order, pₜ can be described by Equation 1, where P_{T} represents transmit power in units of watts (W), G_{T} represents transmit antenna gain in units of decibels relative to isotropic (dBi), A_{T} represents effective aperture area in units of square meters (m2), and λ represents wavelength of the radar signal RF carrier signal in units of meters. In Equation 1, effects of atmospheric attenuation, multi-path propagation, antenna losses, etc. have been neglected.

$${p}_{t} = \frac{{P}_{T}}{4 π {R}^{2}} {G}_{T} = \frac{{P}_{T}}{4 π {R}^{2}} \frac{{A}_{T}}{( {λ}^{2} / 4 π )} = {P}_{T} \frac{{A}_{T}}{{λ}^{2} {R}^{2}} \qquad ( 1 )$$

The transmit power density impinging onto the surface of the target will reflect into the form of reflections depending on the material composition, surface shape, and dielectric behavior at the frequency of the radar signal. Note that off-direction scattered signals are typically too weak to be received back at the radar receiver (such as receive antenna 416 of FIG. 4), so typically, only direct reflections will contribute to a detectable receive signal. In essence, the illuminated area(s) of the target with normal vectors pointing back at the receiver will act as transmit antenna apertures with directivities (gains) in accordance with corresponding effective aperture area(s). The power of the reflections, such as direct reflections reflected and received back at the radar receiver, can be described by Equation 2, where P_{refl} represents effective (isotropic) target-reflected power in units of watts, Aₜ represents effective target area normal to the radar direction in units of m2, Gₜ represents corresponding aperture gain in units of dBi, and RCS represents radar cross section in units of square meters. Also in Equation 2, rₜ represents reflectivity of the material and shape, is unitless, and has a value between zero and one inclusively ([0, . . . , 1]). The RCS is an equivalent area that scales proportional to the actual reflecting area-squared, inversely proportional with the wavelength-squared, and is reduced by various shape factors and the reflectivity of the material itself. For a flat, fully reflecting mirror of area Aₜ, large compared with λ2, RCS=4πAₜ²/λ². Due to the material and shape dependency, it is generally not possible to deduce the actual physical area of a target from the reflected power, even if the target distance R is known. Hence, the existence of stealth objects that choose material absorption and shape characteristics carefully for minimum RCS.

$${P}_{refl} = {p}_{t} {A}_{t} {G}_{t} ∼ {p}_{t} {A}_{t} {r}_{t} \frac{{A}_{t}}{( {λ}^{2} / 4 π )} = {p}_{t} RCS \qquad ( 2 )$$

The target-reflected power (P_{R}) at the location of the receiver results from the reflected-power density at the reverse distance R, collected over the receiver antenna aperture area. For example, the target-reflected power (P_{R}) at the location of the receiver can be described by Equation 3, where A_{R} represents the receiver antenna effective aperture area in units of square meters. In certain embodiments, A_{R} may be the same as A_{T}.

$${P}_{R} = \frac{{P}_{refl}}{4 π {R}^{2}} {A}_{R} = {P}_{T} \cdot RCS \frac{{A}_{T} {A}_{R}}{4 π {λ}^{2} {R}^{4}} \qquad ( 3 )$$

The target distance R sensed by the radar 400 is usable (for example, reliably accurate) as long as the receiver signal exhibits sufficient signal-to-noise ratio (SNR), the particular value of which depends on the waveform and detection method used by the radar 400 to sense the target distance. The SNR can be expressed by Equation 4, where k represents Boltzmann's constant, T represents temperature, and kT is in units of W/Hz]. In Equation 4, B represents bandwidth of the radar signal in units of Hertz (Hz), F represents receiver noise factor. The receiver noise factor represents degradation of receive signal SNR due to noise contributions of the receiver circuit itself.

$$SNR = \frac{{P}_{R}}{kT \cdot B \cdot F} \qquad ( 4 )$$

If the radar signal is a short pulse of duration T_{P} (also referred to as pulse width), the delay τ between the transmission and reception of the corresponding echo can be expressed according to Equation 5, where c is the speed of (light) propagation in the medium (air).

τ=2R/c (5)

In a scenario in which several targets are located at slightly different distances from the radar 400, the individual echoes can be distinguished as such if the delays differ by at least one pulse width. Hence, the range resolution (OR) of the radar 400 can be expressed according to Equation 6.

ΔR=cΔτ/2=cT_{P}/2 (6)

If the radar signal is a rectangular pulse of duration T_{P}, the rectangular pulse exhibits a power spectral density P(ƒ) expressed according to Equation 7. The rectangular pulse has a first null at its bandwidth B, which can be expressed according to Equation 8. The range resolution OR of the radar 400 is fundamentally connected with the bandwidth of the radar waveform, as expressed in Equation 9.

P(ƒ)˜(sin(πƒT_{P})/(πƒT_{P}))² (7)

B=1/T_{P} (8)

ΔR=c/2B (9)

Although FIG. 4 illustrates one example radar 400, various changes can be made to FIG. 4. For example, the radar 400 could include hardware implementing a monostatic radar with 5G communication radio, and the radar can utilize a 5G waveform according to particular needs. In another example, the radar 400 could include hardware implementing a standalone radar, in which case, the radar transmits its own waveform (such as a chirp) on non-5G frequency bands such as the 24 GHz industrial, scientific and medical (ISM) band. In another particular example, the radar 400 could include hardware of a 5G communication radio that is configured to detect nearby objects, namely, the 5G communication radios have a radar detection capability.

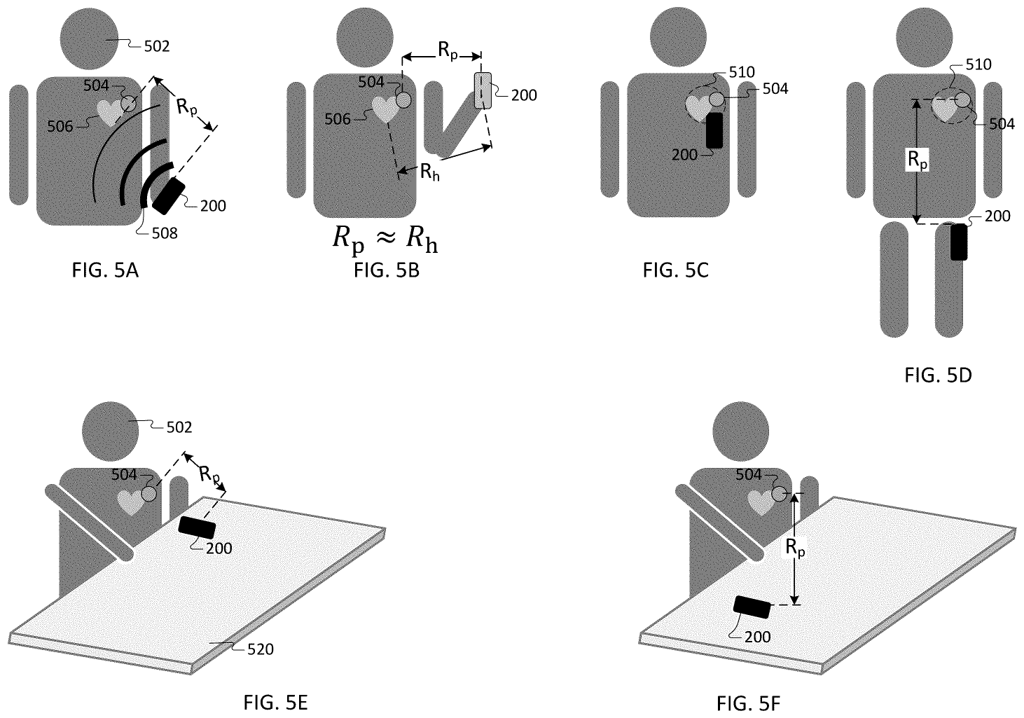

FIGS. 5A-5F illustrates various scenarios in which a pacemaker wearer 502 is the user of the electronic device 200 that executes methods of EMI risk detection and warning, in accordance with an embodiment of this disclosure. As shown in FIGS. 5A-5F, the pacemaker 504 is implanted in the chest near the heart 506 of the user of the electronic device 200.

FIG. 5A illustrates power density of an RF emission 508 from the electronic device 200 dissipating based on a distance from the electronic device 200 to a location of a pacemaker worn by a user of the electronic device, in accordance with an embodiment of this disclosure. The thickest, thinner, and thinnest arced lines demonstrate the power density of the RF emission 508 is stronger closer the body of the electronic device 200 than at a first distance away (e.g., distance from hand to elbow), and is further weaken at second distance away (e.g., distance from hand on a straight arm to heart) farther than the first distance. For ease of illustration, it is understood that the electronic device 200 emits the RF emission 508 in each of the various scenarios shown in FIGS. 5A-5F.

FIG. 5B illustrates a distance R p from the electronic device 200 to a location of a pacemaker worn by a user of the electronic device is approximated as a distance Rₕ from the electronic device to a location of a heart of the user, in accordance with an embodiment of this disclosure.

FIG. 5C illustrates a scenario in which the electronic device 200 detects an EMI risk based on the location of the electronic device 200 positioned inside a location of interest 510 (e.g., inside a shirt pocket) that is close to the heart of a person, in accordance with an embodiment of this disclosure. In this example, the electronic device 200 is positioned inside a front pocket of a shirt worn by a user. The location of interest 510 includes an area close to the heart of a person such that the distance from the pacemaker 504 worn near the location of the heart 506 to the boundary of the location of interest 510 is a short distance. For example, the short distance satisfies a proximity condition. The proximity condition is satisfied when the distance between the pacemaker 504 and the electronic device 200 positioned at least partially inside the location of interest 510 is within a specified range (e.g., closer than a threshold distance). The location of interest 510 can be defined based on the location of a heart of an average person. The threshold distance could depend on the transmission power selected for the RF emission 508. For example, an increased transmission power or greater power density inversely corresponds to a reduced threshold distance.

FIG. 5D illustrates a scenario in which the electronic device 200 does not detect an EMI risk based on the location of the electronic device 200 positioned outside the location of interest 510, in accordance with an embodiment of this disclosure. In this example, the electronic device 200 is positioned inside a pocket of pants worn by a user. The location of the pants pocket includes an area that is not close to the heart 506 of a person. The distance R p between the pacemaker 504 worn near the location of the heart 506 and the electronic device 200 positioned inside the pants pocket is a not a short distance, and does not satisfy the proximity condition due to being (farther than the threshold distance) outside the specified range.

FIG. 5E illustrates a scenario in which the electronic device 200 detects an EMI risk based on the distance R p between the electronic device 200 stationary on a desk 520 and the pacemaker 504 worn by the pacemaker wearer 502 who is (e.g., seated or standing) at the desk 520 being a short distance (e.g., satisfying the proximity condition).

FIG. 5F illustrates a scenario in which the electronic device 200 does not detect an EMI risk based on the distance between the electronic device 200 stationary on a desk 520 and the pacemaker 504 worn by the pacemaker wearer 502 who is (e.g., seated or standing) at the desk 520 being a long distance (e.g., not satisfying the proximity condition).

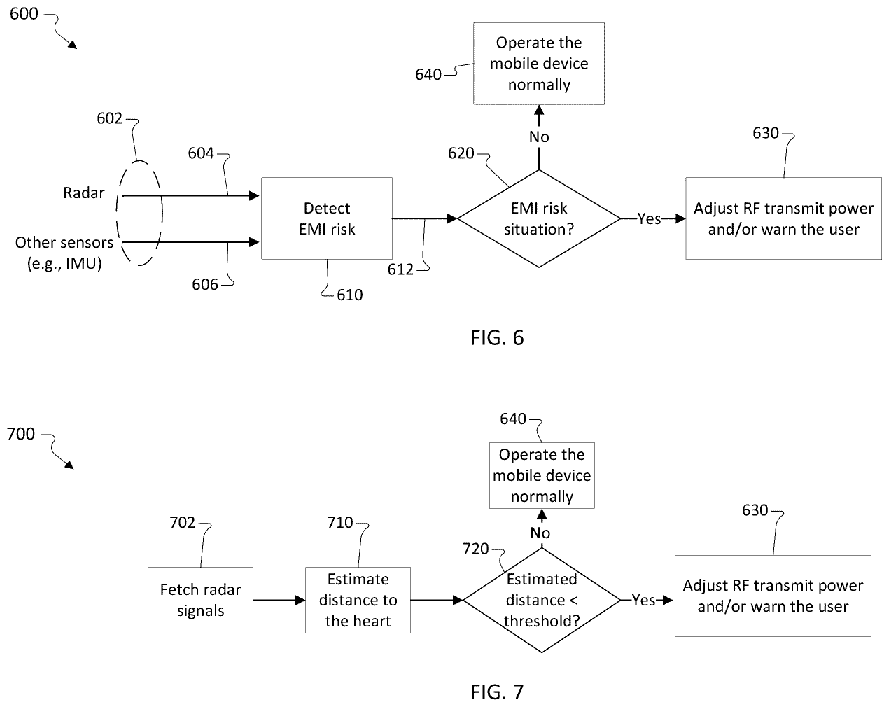

FIG. 6 illustrates a method 600 of EMI risk detection and warning in accordance with an embodiment of this disclosure. The embodiment of the method 600 shown in FIG. 6 is for illustration only. Other embodiments of the method 600 could be used without departing from the scope of this disclosure. For ease of explanation, the method 600 will be described as being implemented in the electronic device 200 of FIG. 2. However, the method 600 could be implemented in any other suitable device.

The method 600 starts when the pacemaker compatibility mode of the electronic device 200 is activated. For example, the processor 240 identifies a setting in which a pacemaker compatibility mode is activated. The method 600 enables the electronic device 200 to automatically detect an EMI risk situation and warn the user.

In the method 600, the electronic device 200 obtains sensor data 602 from a sensor of the electronic device 200. The sensor data 602 can include radar data 604 obtained from a radar, such as the radar 400 of FIG. 4. The sensor data 602 can include other sensor data 606 obtained from other sensors of the electronic device 200, such as inertial measurement unit (IMU) data obtained from the IMU of the electronic device 200. In some embodiments, the sensor data 602 includes both the radar data 604 and the other sensor data 606.

In the method 600, the electronic device 200 operates an electromagnetic interference (EMI) risk detector 610 that detects an EMI risk based on the sensor data 602 obtained. The EMI risk detector 610 generates an indicator 612 that EMI risk to the pacemaker is detected, but does not generate the indicator 612 if the EMI risk detector 610 does not detect an EMI risk.

For example, by analyzing the radar data 604, the EMI risk detector 610 detects whether the field-of-view of the radar includes a heartbeat and estimate a distance from the location of electronic device 200 (as interferer) to the location of the pacemaker. The distance between the pacemaker and the electronic device 200 is estimated based on an assumption that the location of the pacemaker is the location of the heart of the person wearing the pacemaker. The EMI risk detector 610 can output the estimated distance to the heart from the location of the electronic device 200 as the indicator 612 that EMI risk to the pacemaker is detected.

As another example, by analyzing other sensor data 606, the EMI risk detector 610 detects whether the location of the electronic device 200 is in a location of interest (also referred to as an area of interest). The location of interest can be the location of a chest pocket at the front of a shirt or jacket, based on an assumption that the location of the chest pocket is in close proximity to the location of the heart of the person wearing the pacemaker. The EMI risk detector 610 can output an indicator 612 that EMI risk to the pacemaker is detected, in response to a determination that the electronic device is in the location of interest.

In the method 600, the electronic device 200 performs an operation 620 to determine whether a condition for executing an EMI risk mitigation function is satisfied, based on the detected EMI risk. Particularly, in response to the indicator 612 generated by the EMI risk detector 610, the electronic device 200 performs the operation 620. In response to determining that the condition for executing the EMI risk mitigation function is satisfied, the electronic device 200 performs the EMI risk mitigation function 630. The EMI risk mitigation function can include adjusting a radio frequency (RF) transmit power, outputting an alert that warns a user of the electronic device 200 about a pacemaker in relation to the detected EMI risk, or both.

In response to determining that the condition for executing the EMI risk mitigation function is not satisfied, the electronic device 200 performs normal operations 640 without executing the EMI risk mitigation function. The electronic device 200 also performs normal operations 640 without executing an EMI risk mitigation function if the EMI risk detector 610 does not detect an EMI risk (for example, EMI risk detector 610 does not generate the indicator 612).

The EMI risk detector 610 assesses the EMI risk based on the sensor data 602 received from the radar 400 or other sensors 265. If EMI risk detector 610 determined that the situation is not an EMI risk situation, the electronic device 200 may operate normally using the normal transmit power level. If EMI risk detector 610 determined that the situation is an EMI risk situation, then EMI mitigation functions could be performed by the electronic device 200 along with outputting a warning to the user. Those EMI mitigation functions 630 may include terminating RF transmission (at least in certain bands), reducing the transmit power to a safe level (for example, using the distance 412 detected by the radar 400 if the sensor data 602 includes the radar data 604), or switching to operation at high frequency bands such as mmWave. Also, those EMI mitigation functions 630 may include providing a warning to the user, such as by displaying messages (e.g., a pop-up) on the screen of the display 255, or performing a vibration, or outputting a sound notification via the speaker 230. Due to high penetration loss for human tissues, EMI concern for a pacemaker 504 is less at high frequencies. In some embodiments, once the warning has been issued, the electronic device 200 might prompt or request (e.g., require) an input from the user to disable the EMI mitigation functions imposed along with the warning. This option is likely preferable for pacemaker wearers who have more concern about EMI and would want to take extra precaution.

In another implementation, once the electronic device 202 performs the EMI mitigation functions, the EMI risk detector 610 may continue operating until a certain release condition is satisfied (e.g., when consecutive no-EMI-risk is being detected for at least a certain number of times or period of time), then the electronic device 200 would release itself from or cease performance of the EMI mitigation functions imposed. This implementation could allow a non-intrusive operation, such as without displaying a warning to the user or without requesting the input from the user to disable the EMI mitigation functions imposed. While this implementation might provide convenience, there is a tradeoff associated with not requiring the user input to release the EMI mitigation functions imposed. The tradeoff is that the electronic device 200 could stay in the EMI risk situation longer (compared to when the user intervenes), thereby resulting in lower performance (e.g., reduced throughput due to reduce RF transmit power) for the communication systems. In this case, it might still be beneficial to issue the warning, so that the user may take action to move the electronic device 200 outside the EMI risk situation rather than waiting for a natural change in the normal usage by the user (which could take a very long time). Note that by outputting a warning, the electronic device 200 also helps remind the user that an EMI risk situation occurred and helps the user learn to avoid and prevent EMI risk situations in the future.

Although FIG. 6 illustrates one example method 600 of EMI risk detection and warning, various changes can be made to FIG. 6. For example, each of FIGS. 7-14 illustrate a variation of the method of EMI risk detection and warning in accordance with an embodiment of this disclosure. The embodiments of the methods shown in FIGS. 7 and 10-14 are for illustration only. Other embodiments of the methods 700, 1000, 1100, 1200, 1300, and 1400 could be used without departing from the scope of this disclosure. For ease of explanation, the methods 700, 1000, 1100, 1200, 1300, and 1400 will be described as being implemented in the electronic device 200 of FIG. 2, but could be implemented in any other suitable device. Each of the methods shown in FIGS. 7 and 10-14 is executed while the pacemaker compatibility mode of the electronic device 200 is activated.

FIG. 7 illustrates an example method 700 of direct EMI risk detection and warning that uses radar data to directly estimate the distance to the heart to determine if an EMI risk situation occurs, in accordance with an embodiment of this disclosure. The method 700 can be implemented in situations, such as shown in FIGS. 5A, 5B, 5E, and 5F. To avoid duplicative descriptions, the EMI risk mitigation function 630 and the normal operations 640 described with FIG. 6 can be the same or similar as shown in FIG. 7.

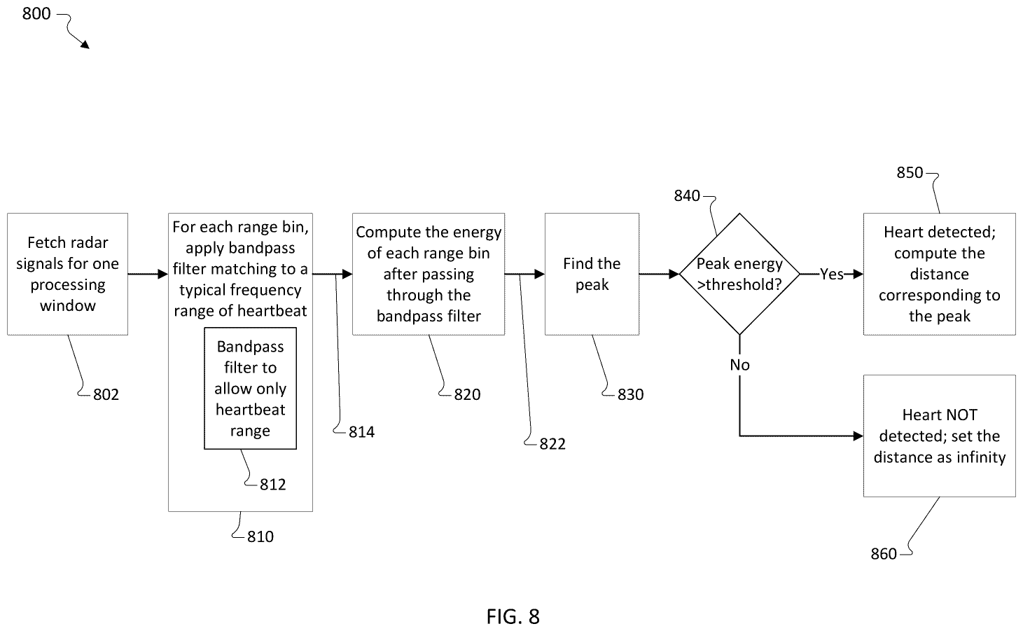

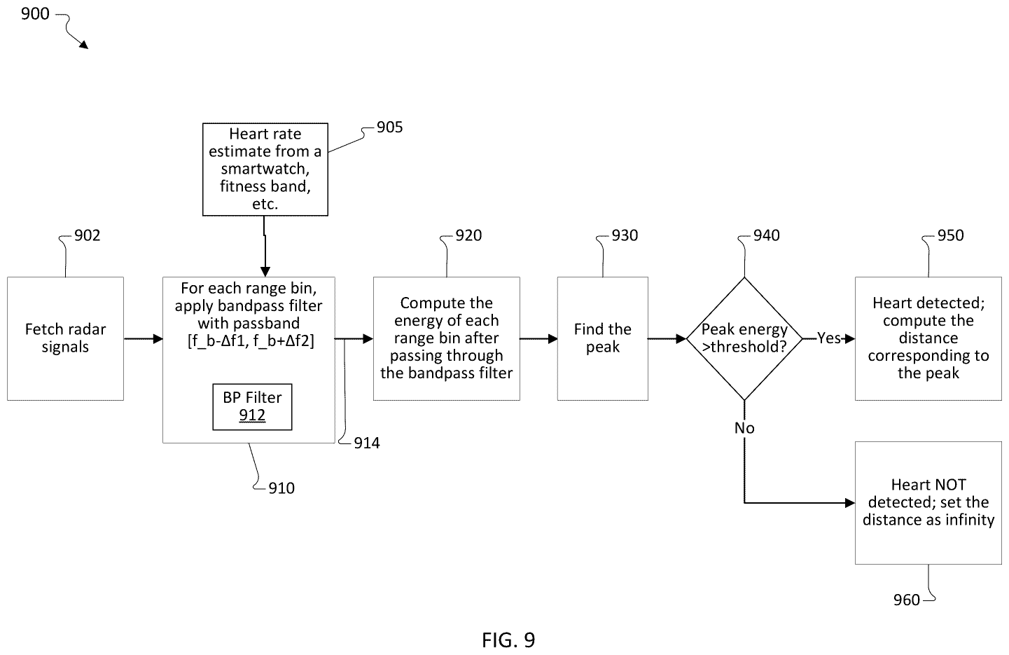

At block 702, the electronic device 200 obtains radar data 604 in the form of radar signals from the radar 400. The radar signal corresponds to a sliding input data window W (also referred to as a processing window W) that includes recent radar frames from the radar data 604. At block 710, a distance Rₕ to the heart 506 is estimated. The procedure performed at block 710 can be performed by the EMI risk detector 610 of FIG. 6, and can include the methods of FIG. 8 or FIG. 9. At block 720, the estimated distance Rₕ to the heart is compared to a threshold distance. Particularly, electronic device 200 determines whether the estimated distance Rₕ to the heart satisfies a proximity condition, which is satisfied if the distance Rₕ to the heart is within a specified range (Rₕ<threshold distance). The procedure performed at block 720 can be the same as or similar to the procedure performed at operation 620 of FIG. 6. If the proximity condition is satisfied, then the method 700 proceeds to perform the EMI risk mitigation function 630. However, if the proximity condition is not satisfied, then the method 700 proceeds to the normal operations 640.

The embodiment of the method 700 shown in FIG. 7 uses radar data 604 to estimate the distance Rₕ to the heart 506 to determine whether the electronic device 200 is in an EMI risk situation. The determination of an occurrence of an EMI risk situation is based on the distance between the electronic device 200 (which in this case is the interferer) and the pacemaker 504. The location of the pacemaker 504 is approximated by the heart 506 of the user. The radar data 604 obtained at block 702 is used by the electronic device to detect and measure the distance Rₕ from the mobile device to the heart 506 instead of measuring an actual distance to the pacemaker 504. If the estimated distance Rₕ to the heart is less than the threshold distance, then at block 720, the electronic device 200 is determined to be in an EMI risk situation, and the electronic device 200 performs the EMI risk mitigation function 630 of adjusting the transmit power and/or outputting a warning.

FIG. 8 illustrates an example method 800 of estimating the distance to the heart using radar data, in accordance with an embodiment of this disclosure. The method 800 can be performed at block 710 of FIG. 7. The embodiment of the method 800 shown in FIG. 8 is for illustration only, and other embodiments could be used without departing from the scope of this disclosure. For ease of explanation, the method 800 will be described as being implemented in the electronic device 200 of FIG. 2, but could be implemented in any other suitable device. To avoid duplicative descriptions, the procedures performed at block 702 described with FIG. 7 can be the same as or similar to the procedure performed at corresponding block 802 of FIG. 8.

At block 810, for each range bin, the electronic device 200 applies a bandpass filter 812 matching to a typical frequency range of a heartbeat. The bandpass filter 812 is used to filter out irrelevant signal energy (i.e., those that do not correspond to the beating heart 506). The bandpass filter cutoff frequencies are selected to allow a typical range of frequencies corresponding to typical range of human's heart rate, for example, a range of 40-200 beats per minute.

The filtered radar data 814 output from the bandpass filter 812 can be interpreted as the energy reflected by the heart. At block 820, the electronic device 200 computes the energy of each range bin after passing through the bandpass filter.

At block 830, a peak energy is determined from the energy of each range bin. That is, a peak detection function is executed to determine which range bin has the highest reflected power from the heart. To avoid false alarm due to noise, first the energy at the peak is compared against a detection threshold, namely a threshold energy value. For example, this threshold energy value may be set to 3 dB from the noise floor, where the noise floor may be estimated by computing the median across the computed energy 822 of each range bin after the bandpass filtering.

At block 840, the electronic device determines whether the determined peak energy satisfied a threshold energy condition. The threshold energy condition is satisfied if the determined peak energy is within a specified energy range (e.g., determined peak energy is greater than a threshold energy value).

At block 850, in response to a determination that the threshold energy condition is satisfied, a heart is detected, and the electronic device 200 computes the distance Rₕ to the heart corresponding to the determined peak energy. The distance Rₕ to the heart is estimated by the distance of the range bin of the peak.

At block 860, in response to a determination that the threshold energy condition is not satisfied, a heart is not detected, and the electronic device 200 sets the distance Rₕ to the heart 506 as a large value (e.g., infinity).

The principle for detecting the heart using radar is based on the observation that the heart movement causes a slight perturbation (e.g., movement) in the chest surface, with typical displacement in the order of millimeters. While this is a small perturbation, the radar 400 can track the phase changes of such a target (e.g., chest perturbation) and detect this kind of minor movement accurately and reliably. The typical range of frequencies that a human heart may beat can be a design parameter configured into the pacemaker compatibility module 263. At block 810, the electronic device 200 filters out irrelevant frequencies outside that range of frequencies, allowing the filtered radar data to be used to detect the location of the heart 506. In a first embodiment of direct EMI risk detection, the method 800 of FIG. 8 uses radar data 604 only. In second embodiment of direct EMI risk detection, which is described more particularly below, the method 900 of FIG. 9 uses the radar data 604 and heart rate estimation information received from an external electronic device (referred to as a “helper device” such as a smart watch or fitness band) that has heart rate measurement capability.

Depending on the choice of the range estimation solution, some further fine tuning like interpolation may also be used. Such an interpolation might be useful when the radar 400 does not use a large bandwidth (thus, will have poor range resolution). With a large enough bandwidth (such as 5 GHz) used by the radar 400, the range resolution can be approximately 3 cm, and likely there is no need for such interpolation for the main use cases described in this disclosure.

FIG. 9 illustrates an example method 900 of estimating the distance to the heart using radar data and an external electronic device that has heart rate measurement capability (e.g., a helper device such as a smart watch or fitness band) that provides a heart rate measurement, in accordance with an embodiment of this disclosure. The method 900 can be performed at block 710 of FIG. 7. The embodiment of the method 900 shown in FIG. 9 is for illustration only, and other embodiments could be used without departing from the scope of this disclosure. For ease of explanation, the method 900 will be described as being implemented in the electronic device 200 of FIG. 2, but could be implemented in any other suitable device. The helper device will be described as being implemented by a smart watch that is worn by and that has registered the same user as the user of the electronic device 200. To avoid duplicative descriptions, the procedures performed at block 802, 820, 830, 840, 850, and 860 described with FIG. 8 can be the same as or similar to the procedure performed at corresponding blocks 902, 920, 930, 940, 950, and 960 of FIG. 9.

At block 910, the electronic device 200 receives heart rate estimation information from a helper device 905 that is an external electronic device that has heart rate measurement capability. The helper device 905 is worn by the user of the electronic device 200, and may be paired to communicate with the electronic device, or may be connected to a shared user account. Also at block 910, for each range bin, the electronic device 200 applies a bandpass filter 912 matching to a typical frequency range of a heartbeat. The heart rate measurement received from the helper device 905 is used by the electronic device 200 to customize the bandpass filter, which is a technical advantage that makes the estimation of the distance Rₕ to the heart more reliable.

This heart rate estimate received from the helper device 905 is used to fine tune the bandpass filter 912, to narrow down the cutoff frequencies of the filter 912 to be tailored specifically to the current heart rate of the user, as opposed to a typical human. This fine tuning is a technical solution that reduces the amount of irrelevant signal energy (which could be noise or other interfering signals) in the filtered radar data 914 that is output from the bandpass filter 912 before the peak detection is performed. This fine tuning is a technical solution that improves the SNR and contributes to improvements in the detection reliability in the method 900 of FIG. 9, compared to the embodiment of FIG. 8, which uses only radar data 604 and which is based on an assumption that provides a wider bandwidth of the bandpass filter 812.

Particularly in the example filter 912 shown in FIG. 9, ƒ_b denotes the heart rate estimate received from the helper device 905, and ƒ_b-Δƒ1 and ƒ_b-Δƒ2 are the cutoff frequencies of the bandpass filter 912, which could be selected to account for the heart rate estimation error range of the helper device 905. Δƒ1 and Δƒ2 denote adjustments for inaccuracy of the helper device 905, for example, the heart rate estimation error range.

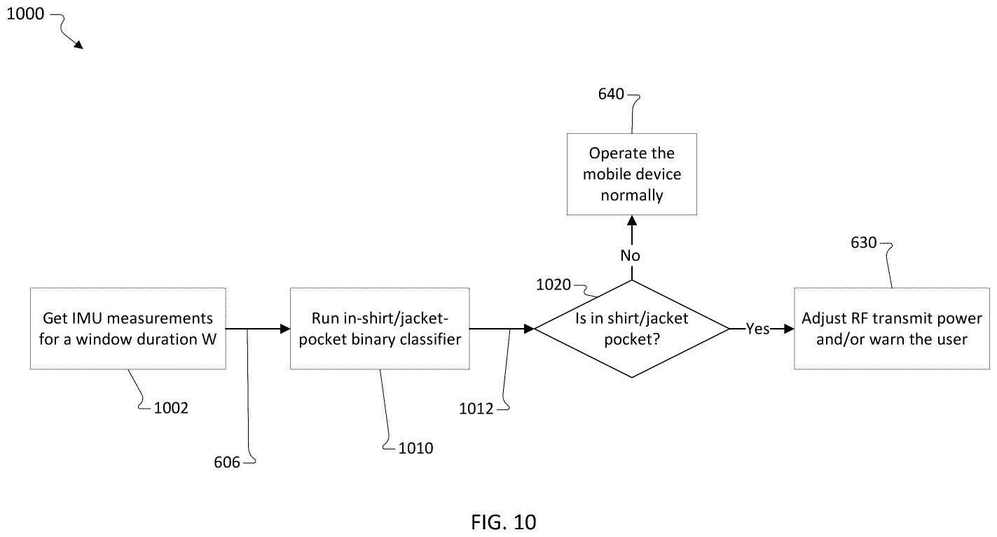

FIG. 10 illustrates an example method 1000 of indirect EMI risk detection and warning that uses sensor data to indirectly determine if an EMI risk situation occurs, in accordance with an embodiment of this disclosure. That is, the method 1000 uses sensor data to indirectly determine if the electronic device 200 is in an EMI risk situation. The method 1000 can be implemented in situations, such as shown in FIGS. 5C and 5D. To avoid duplicative descriptions, the EMI risk mitigation function 630 and the normal operations 640 described with FIG. 6 can be the same or similar as shown in FIG. 10.

As a comparison, the methods of FIGS. 7-9 describe direct detection of an EMI risk situation using radar to estimate the distance from the mobile device to the heart, but the method 1000 of FIG. 10 describes an indirect detection of a particular EMI risk situation, which is the case when the electronic device 200 is placed in the user's shirt/jacket pocket.

At block 1002, the electronic device 200 obtains other data 606 from other sensors 265, including IMU measurements that correspond to a processing window W. One set of relevant sensors 265 are motion sensors such as IMU, gyroscope, accelerometer, etc. For simplicity of illustration, only IMU is shown in FIG. 10, but it is understood that other sensors such as a gyroscope, accelerometer, etc. may also be used.

The method 1000 includes executing a binary classifier 1010 that classifies that other sensor data 606 (e.g., IMU data) as belonging to a first class indicating that electronic device is IN the location of interest 510 (e.g., in the shirt pocket) or as belonging to a second class indicating that the electronic device 200 is OUT of the location of interest 510. The binary classifier 1010 outputs a prediction 1012. At block 1020, the electronic device determines whether the electronic device 200 is in the shirt pocket based at least in part on the prediction 1012 output from the classifier. For example, if the prediction 1012 is the first class, then a determination that the electronic device 200 is inside the shirt pocket is made, and the method proceeds to perform the EMI risk mitigation function 630. If the prediction 1012 is the second class, then a determination that the electronic device 200 is outside the shirt pocket is made, and the method proceeds to the normal operations 640.

The method 1000 is based on an observation that when the electronic device 200 is placed in a shirt/jacket pocket, while the user is walking, the sensors 265 capture signals and IMU measurements matching a particular signature due to the device placement in the shirt/jacket pocket. For example, for a normal shirt that is not tightly fitted to the body, the electronic device 200 in the pocket would sway back and forth as the user moves or walks. The electronic device 200 utilizes a pattern recognition algorithm that identifies the pattern (i.e., the particular signature due to the device placement in the shirt/jacket pocket of a person walking) in a sequence of sensor measurements to determine if the electronic device 200 is likely placed in a shirt or jacket pocket.

If other sensors are also available, such other sensors could be used in combination with the motion sensors that could help improve the accuracy of the in shirt/jacket pocket classification (e.g., determined at blocks 1010 and 1020 collectively). These other sensors may also include radar. For example, proximity sensor or radar can detect if the electronic device 200 is likely in an enclosed space (which is a necessary condition for in-pocket classification). Similarly, light sensor can measurement brightness, which can be used as an indicator that the device 200 is likely in an enclosed space (i.e., darkness inside the pocket). In one example, the light sensor reading could be added as another input to the pattern recognition algorithm that may be based on a machine learning (ML) system.

In another example, the light sensor measurement may be used to apply a gating rule on the prediction 1012 by the classifier. Specifically, if the classifier prediction 1012 is the first class (‘in-shirt/jacket-pocket’), then the light sensor measurement is fetched and compared against a threshold. If light sensor measurements are greater than the threshold (i.e., brighter than expected), then it is determined that electronic device 200 is not in an enclosed space (e.g., expected to be a dark space), and thus it is unlikely to be in a pocket. The prediction 1012 output is overwritten as the second class (‘Not in-shirt/jacket-pocket’).

In some embodiments, the binary classifier 1010 may be fine-tuned online if feedback from the user is available. One such a case is when the explicit input from the user (e.g., by clicking a button) is needed for disabling the warning. In such a situation, the interface for the input from the user may provide a choice to indicate if this warning event is a false alarm (i.e., the device is not in a shirt/jacket pocket and not an EMI risk). This provides additional labeled training data that could be used to fine-tune or retrain the classifier so that the solution is more customized for the user with better performance than the generic solution. Such a fine-tuning or retraining procedure may be conducted once the number of feedbacks collected is larger than some threshold.

Because typical sensors on an electronic device (such as the IMU) can only capture a partial picture of the status of the device, some confusions (which leads to false alarm or misdetection) cannot be avoided. As described above, some temporal filtering as well as some gating rules (such as based on the light sensor) could be incorporated to reduce such occurrences of false alarms, though it cannot be avoided 100%. Depending on the concern level of the user of EMI, false alarm or misdetection could be prioritized. If the user's concern level is high, some false-alarm is tolerable, but misdetection should be minimized. On the other hand, if the user's concern level is nominal (i.e., not as high), a more balanced choice could be used. Such adjustment could be made using a metric of the confidence level from the pattern recognition, such as the binary classifier 1010.

The prediction 1012 output is often in the form of a probability. For example, focusing on the first class (i.e., in-shirt/jacket-pocket), by setting the probability threshold to a high value (such as 0.9 in the case of a high concern level), then an occurrence of a false alarm will be reduced compared to a neutral value (such as 0.5 in the case of a nominal concern level) as the threshold choice.

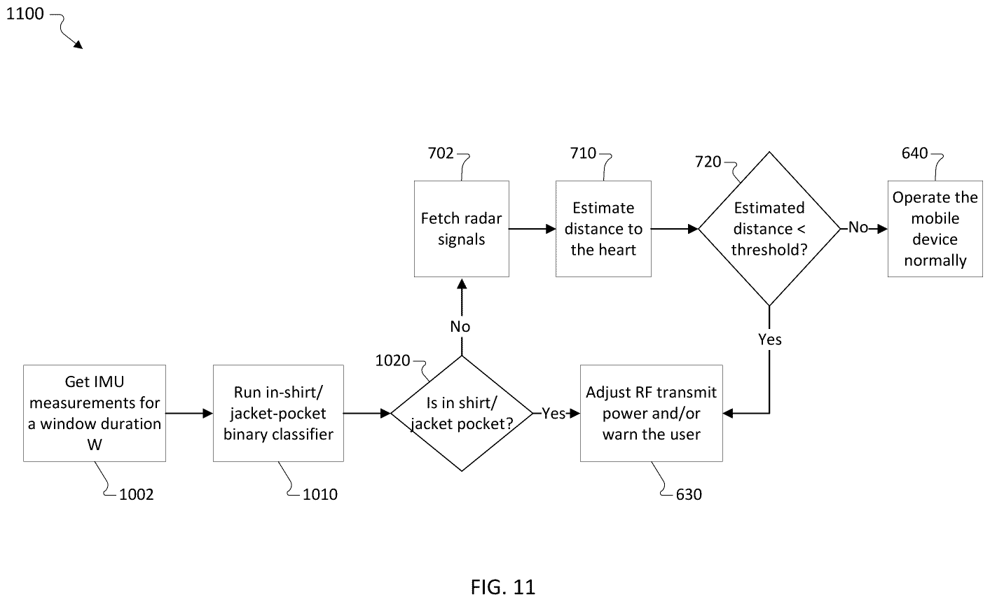

FIG. 11 illustrates an example method 1100 of multiple-stage EMI risk detection and warning including a primary stage in which a primary sensor is used to indirectly determine if an EMI risk situation occurs, and a secondary stage in which the radar is subsequently activated as a secondary sensor to directly determine if the EMI risk situation occurs, in accordance with an embodiment of this disclosure. More particularly, FIG. 11 shows the example method 1100 of multiple-stage EMI risk detection and warning that, in response to a determination that an EMI risk situation is not indirectly detected using sensor data from a primary sensor, activates radar as a secondary sensor to directly estimate the distance to the heart. To avoid duplicative descriptions, the EMI risk mitigation function 630 and the normal operations 640 described with FIG. 6 can be the same or similar as shown in FIG. 11. The procedures performed at block 702, 710, and 720 described with FIG. 7 can be the same as shown in FIG. 11. The procedures performed at block 1002 and the binary classifier 1010 described with FIG. 10 can be the same as shown in FIG. 11.

In the method 1100, the default or primary stage of EMI risk detection is based on the indirect detection method. When an EMI risk is not detected by the primary indirect method, then the radar 400 is activated to perform the radar-based direct detection method as the secondary stage EMI risk detection. This embodiment provides technical advantages, including cost reduction due to not operating the radar by default. That is, in this embodiment, because the direct detection using the radar 400 is only used when needed, the cost for operating the radar (including the transmission power as well as the computational cost for the processing of radar signals) can be saved. At block 1120, the procedure performed is similar to the procedure performed at block 1020 of FIG. 10, except that the electronic device 200 activates the radar 400 in response to a determination that an EMI risk is not detected by the primary indirect method, and the method 1100 proceeds to block 702.

Compared to the direct detection methods and the indirect detection methods for detecting an EMI risk situation, the method 1100 of FIG. 11 combines these two indirect and direct methods to complement each other and improve the overall detection performance. For example, when the electronic device 200 is placed in a pocket, depending on the clothing material, the radar signal may or may not penetrate the clothing material well. If radar signals can only poorly penetrate the clothing material, then the radar 400 would not be able to directly estimate the distance Rₕ to the heart due to the blockage by the cloth.

In a different scenario, when the electronic device 200 is placed on a surface such as on a desk (e.g., desk 520 of FIG. 5), which the user is sitting close to the desk, only the direct detection using radar can determine whether the device 200 is in an EMI risk situation or not. If the electronic device 200 stationary on the desk 520, then the other sensor data 606 will not measure movement and will not generate a pattern matching the particular signature due to the device placement in the shirt/jacket pocket of a person walking.

If the binary classifier 1010 determines that the electronic device 200 is in the shirt/jacket pocket, then an occurrence of an EMI risk situation is determined. In this case, there is no need to activate the radar 400, and the method 1000 proceeds to performance of the EMI risk mitigation function 630 of providing a warning to notify the user, while at the same time, the electronic device 200 can adjust the RF transmission power level.

In the case when the binary classifier 1010 determines that the electronic device 200 is not in-shirt/jacket pocket, then it could still be possible that the device 200 might still be placed in proximity to the heart of the user. For example as shown in FIG. 5E, when the user is working at a desk while having the electronic device 200 nearby, the distance R p could be a (close) short distance, such as less than 20 or 30 cm. That is, the electronic device 200 could still be an occurrence of an EMI risk situation. To eliminate this kind of misdetection, in case of the second class (i.e., not in-shirt/jacket pocket) as the prediction 1012 from the binary classifier 1010, the method 1100 further executes the direct detection using the radar 400.

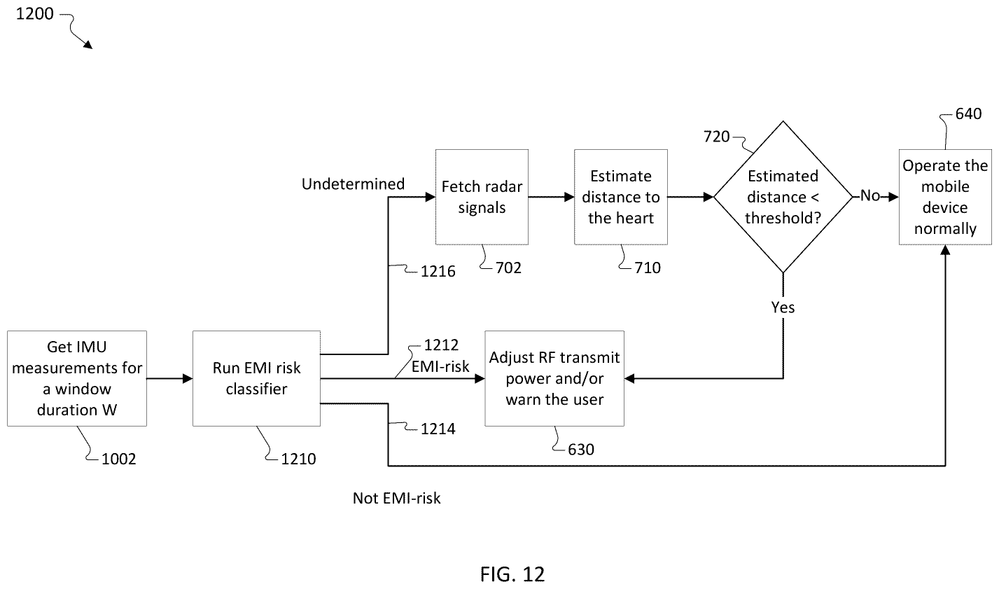

FIG. 12 illustrates an example method 1200 of EMI risk detection and warning that includes a ternary classifier 1210 that differentiates three classes and activates radar-based direct detection of an EMI risk situation based on an “undetermined” classification from among the three classes, in accordance with an embodiment of this disclosure. To avoid duplicative descriptions, the EMI risk mitigation function 630 and the normal operations 640 described with FIG. 6 can be the same or similar as shown in FIG. 12. The procedures performed at block 702, 710, and 720 described with FIG. 7 can be the same as shown in FIG. 12. The procedures performed at block 1002 described with FIG. 10 can be the same as shown in FIG. 12.

The method 1200 executes a ternary classifier 1210 instead of using a binary classifier (such as 1010 of FIG. 10) for the indirect EMI risk detection. The ternary classifier 1210 differentiates three classes, which may include a first class for EMI risk situation (e.g., in shirt/jacket pocket), a second class for not EMI risk situation (e.g., when the device is in a pants-pocket), and another third class for an undetermined situation (e.g., when the device is static such as when placed on a desk). The ternary classifier 1210 outputs a first, second, or third prediction 1212, 1214, 1216 corresponding to the first, second, and third classes, respectively.

This disclosure describes at least two embodiments for implementing this ternary classifier 1210. The first embodiment of the ternary classifier 1210 uses the same training dataset as the binary classifier 1010, but employs an open-set recognition solution. Open-set recognition solutions are techniques to train a classifier (such as the ternary classifier) so that after the training, the classifier is able to output the third prediction 1216 of “unknown” or “undetermined” classification for classes that were not present in the training set. A simple technique uses the prediction probability (e.g., the threshold could be set to a relatively high value such as 0.7, and anything below that is output as unknown.).

The first embodiment of the ternary classifier 1210 introduces a new, third class explicitly as unknown (for the undetermined situation). In this case, the classes can be defined as follows: a first class (labeled “EMI-risk”) indicating the device 200 is in the shirt/jacket-pocket; a second class (labeled “Not EMI-risk”) indicating the device 200 is in the pants-pocket (back or front pocket of pants) and outside the location of interest 510; and a third class (labeled “Undetermined”) indicating a static situation such as when the device 200 is stationary on a desk and the pacemaker 504 worn by the user may be close or far). The third class of undetermined can indicate a hand-holding situation, such as when using the device 200 during a phone call while holding the device on the left ear may be an EMI risk but not on the right ear. The third class of undetermined can indicate other random use cases not belonging to the first two classes.

Note that these two embodiments of the ternary classifier 1210 may be combined and used together. When used together, the unknown by the open-set solution and when classified as undetermined situation are treated the same, that is as an undetermined situation. With this richer ternary classifier 1210, the radar 400 is activated for direct detection of an EMI risk situation only for the undetermined situation when the third prediction 1216 is output. This ternary classifier 1210 provides technical advantages including further reducing the cost for operating the radar 400 compared to the embodiment of FIG. 11.

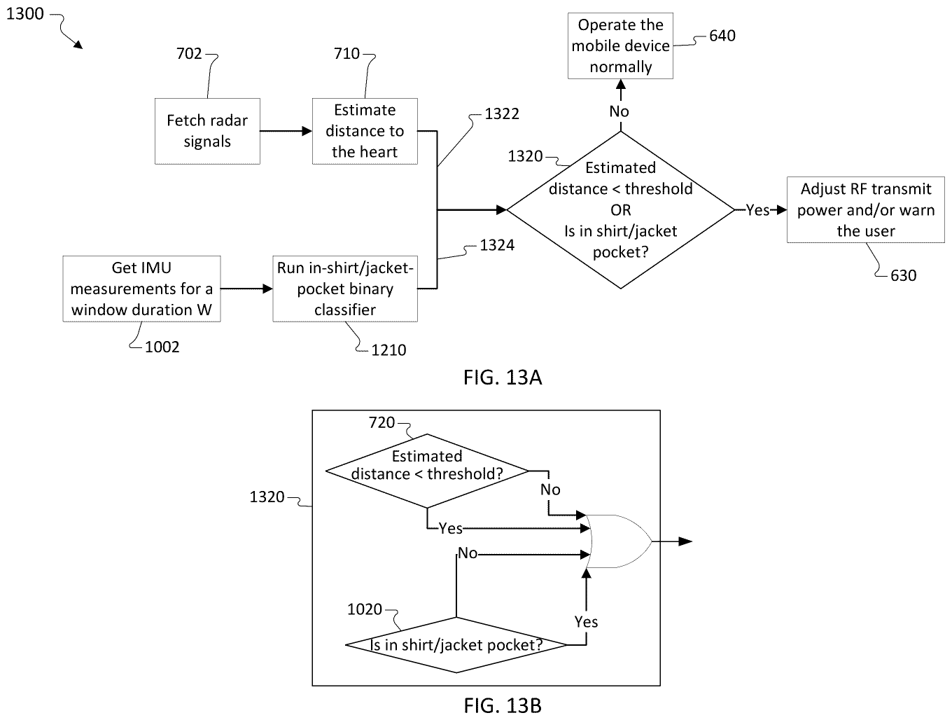

FIG. 13A illustrates an example method 1300 of direct/indirect hybrid EMI risk detection and warning that concurrently (for example, simultaneously) uses both radar-based direct detection of an EMI risk situation and indirect detection of the EMI risk situation based on other sensors, in accordance with an embodiment of this disclosure. FIG. 13B illustrates a detailed view of components within block 1320 of FIG. 13A. FIGS. 13A and 13B are collectively referred to as FIG. 13. To avoid duplicative descriptions, the EMI risk mitigation function 630 and the normal operations 640 described with FIG. 6 can be the same or similar as shown in FIG. 13. The procedures performed at block 702, 710, and 720 described with FIG. 7 can be the same as or similar shown in FIG. 13. The procedures performed at block 1002 and ternary classifier 1210 described with FIG. 12 can be the same as shown in FIG. 13.

At block 1320, the electronic device 200 determines whether a condition for executing an EMI risk mitigation function is satisfied, based on a result 1322 that EMI risk is directly detected based on radar data 604 and based on another result 1324 that EMI risk is indirectly detected based on other sensor data 606 (IMU data). In some embodiments of block 1320, the main goal is to avoid misdetection of an EMI risk situation, such as when the user selects a high concern level, and a design choice is to execute a logical OR gate operation on the detection results from the direct and indirect detection solutions, namely, the output from block 720 and 1020, respectively. That is, block 1320 includes block 720 and 1020 of FIGS. 7 and 10, respectively.

In the method 1300, the cost of operating the radar 400 is not a design concern, and direct and indirect EMI risk detection modules are operated simultaneously. This method 1300 provides a higher detection accuracy than other embodiments of this disclosure. For example, without any movement, the ternary classifier 1210 might not be reliable to distinguish between in-shirt-pocket (EMI risk) or in-pants-pocket (not EMI risk) or in-bag (undetermined) situation. The radar-based direct detection technique using the radar 400 can help close some of these gaps or improve detection accuracy. For example, depending on the material of the shirt/jacket, radar signals may not have much penetration loss. For such materials, especially when the user has little to no motion (e.g., like sleeping), the radar 400 could still measure the distance Rₕ to the heart and make a determination whether the electronic device 200 is in an EMI risk situation or not.

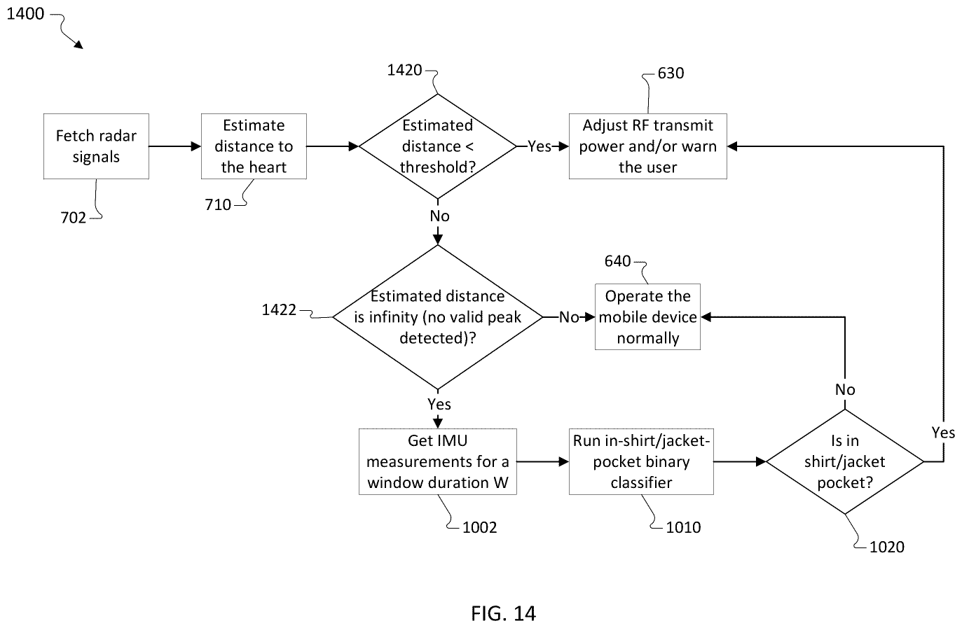

FIG. 14 illustrates an example method 1400 of multiple-stage EMI risk detection and warning including a primary stage in which radar-based direct detection of an EMI risk situation is performed, and a secondary stage in which a secondary sensor is subsequently used to indirectly determine if the EMI risk situation occurs, in accordance with an embodiment of this disclosure. To avoid duplicative descriptions, the EMI risk mitigation function 630 and the normal operations 640 described with FIG. 6 can be the same or similar as shown in FIG. 14. The procedures performed at block 702 and 710 described with FIG. 7 can be the same as shown in FIG. 14. The procedures performed at block 1002, the binary classifier 1010, and block 1020 described with FIG. 10 can be the same as shown in FIG. 14.

In the method 1400, the radar-based direct EMI detection technique is used as the primary stage of EMI detection. When the direct detection by radar fails, then the indirect EMI detection technique is used as the secondary stage. As an example, the direct detection by radar fails when peak energy is below the detection threshold, in which case the estimated distance Rₕ is set to the large value (such as infinity).

In some embodiments, the method 1400 includes the binary classifier 1010 of FIG. 10 for the in-shirt/jacket-pocket is used. In other embodiments, the method 1400 includes the richer ternary classifier 1210 of FIG. 12 that can classify an undetermined situation. In the embodiments with the ternary classifier 1210, the radar 400 struggles to detect the heart, so a design choice may classify the undetermined situation such that the second prediction 1214 (labeled “not EMI-risk”) is output.

The procedure performed at block 1420 is similar to the procedure performed at block 720 of FIG. 7, except that the method proceeds to block 1422 in response to a determination that the proximity condition is not satisfied. At block 1422, the electronic device 200 determines whether the estimated distance Rₕ to the heart is set to the large value (e.g., infinity). For example, at block 1422, it is identified whether no valid peak was detected (such as block 860 of FIG. 8) such that the electronic device 200 determined a heart 506 was not detected. In response to a determination that the estimated distance Rₕ to the heart is set to the large value, the method 1400 proceeds to block 1002. In response to a determination that the estimated distance Rₕ to the heart is not set to the large value, the method 1400 proceeds to the normal operations 640.

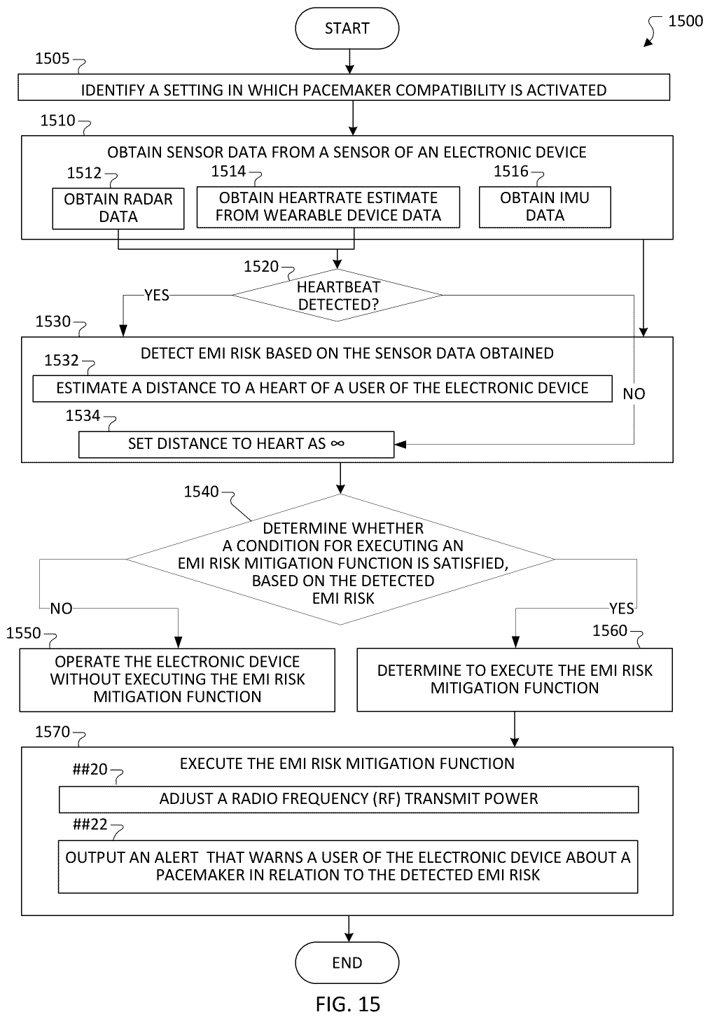

FIG. 15 illustrates a method 1500 for warning a wearer of a pacemaker against potential electromagnetic interference by a radar-aided mobile device, in accordance with an embodiment of this disclosure. The embodiment of the method 1500 shown in FIG. 15 is for illustration only, and other embodiments could be used without departing from the scope of this disclosure. The method 1500 is implemented by an electronic device, such as the electronic device 200 of FIG. 2 that includes the radar 400 of FIG. 4. More particularly, the method 1500 could be performed by a processor 240 of the electronic device 200 executing the pacemaker compatibility module 263. For ease of explanation, the method 1500 is described as being performed by the processor 240.

At block 1505, the processor 240 identifies a setting in which pacemaker compatibility mode is activated. If the processor 240 pacemaker identifies an opposite setting (i.e., alternative setting) in which the pacemaker compatibility mode is deactivated, the method 1500 ends.

At block 1510, the processor 240 obtains sensor data 602 from a sensor of an electronic device. In certain embodiments as shown at block 1512, the processor 240 obtains the sensor data 602 by obtaining radar data 604 into a sliding input data window W that includes recent radar frames from the radar data 604. The sensor data 602 can include both the radar data 604 and the other sensor data 606. In certain embodiments as shown at block 1514, the processor 240 receives heart rate estimation information from an external electronic device (i.e., helper device 905) worn by a user of the electronic device 200.

In certain embodiments as shown at block 1516, the processor 240 obtains the sensor data 602 by obtaining IMU data corresponding to a sliding input data window W. The other sensor data 606 includes the obtained IMU data. In certain embodiments, the method 1500 includes classifying the IMU data as one from among a set of EMI risk classes. In embodiments that include a binary classifier, the set of EMI risk classes include: a first class indicating that a location of the electronic device is IN a location of interest 510, and a second class indicating that the location of the electronic device is OUT of the location of interest 510. In embodiments that includes a ternary classifier, the set of EMI risk classes includes the first class, the second class, and a third class indicating that the location of the electronic device 200 is UNDETERMINED relative to the location of interest 510. In such embodiments, processor 240, in response to the IMU data being classified as the third class (i.e., UNDETERMINED as shown in FIG. 12), the method 1500 proceeds to block 1530 at which the processor 240 detects the EMI risk by activating a radar 400 of the electronic device 200, based on the IMU data classified as the third class.

The method 1500 includes block 1520 in embodiments in which the obtained sensor data includes radar data 604 or a heartrate estimate. At block 1520, the processor 240 determines whether a heartbeat of the user of the electronic device 200 is detected. In response to a determination that a heartbeat is not detected, the method 1500 proceeds to block 1534 at which the processor 240 sets the distance Rₕ to the heart 506 as a large value (e.g., infinity) that is significantly greater than the threshold distance that defines the proximity condition.

For example, processor 240 determines that the heartbeat of the user is detected in response to receipt of wearable device data that includes a heartrate estimate. In some embodiments, processor 240 filters (e.g., using a bandpass filter of FIG. 8) the data window W to pass through a subset of the radar data that matches a typical frequency range of a heartbeat, and detects that a heartbeat is present based on a determination that an energy of the subset of the radar data satisfies a threshold energy condition. In some embodiments, the processor 240 determines the typical frequency range of the heartbeat based on the heart rate estimation information received from the helper device 905 that is worn by the user.

At block 1530, the processor 240 detects EMI risk based on the sensor data obtained. In embodiments in which the pace maker compatibility mode is not always activated, but can be selectively deactivated or activated, the processor 240 detects the EMI risk based on identifying a setting in which pacemaker compatibility is activated. More particularly, the processor 240 determines whether the electronic device 200 is in an EMI risk situation such that the electronic device is an interferer causing EMI risk to a pacemaker 504 worn by a user of the electronic device 200. In certain embodiments as shown at block 1532, the processor 240 detects the EMI risk by estimating a distance to a heart 506 of a user of the electronic device 200.

At block 1540, the processor 240 determines whether a condition for executing an EMI risk mitigation function is satisfied, based on the detected EMI risk. In some embodiments, the processor 240 determines whether the condition for executing an EMI risk mitigation function is satisfied by determining whether the distance to the heart satisfies a proximity condition. The proximity condition is satisfied if the distance Rₕ to the heart of the user (as estimated at block 1532 or as set at block 1534) is within a specified range. The specified range can be defined by a threshold distance, such as less than the threshold distance. The distance R p between the electronic device 200 and the pacemaker 504 is set as equivalent to the distance Rₕ to the heart of the user. Accordingly, the proximity condition is satisfied if the distance R p to the pacemaker 504 is within the specified range, but the proximity condition is not satisfied if the distance R p to the pacemaker 504 is outside the specified range, such as greater than or equal to the threshold distance.

In some embodiments, the processor 240 determines whether the condition for executing an EMI risk mitigation function is satisfied by determining whether the other sensor data 606 indicates that the electronic device 200 is in a location of interest 510 relative to the heart 506 of the user. Particularly, the processor 240 determines whether IMU measurements obtained from the IMU indicate that the location of the electronic device 200 is at least partially within the location of interest 510.

In response to determining that the condition for executing the EMI risk mitigation function is not satisfied, the method 1500 proceeds to block 1550, at which the processor 240 operates the electronic device 200 without executing the EMI risk mitigation function. In response to determining that the condition for executing the EMI risk mitigation function is satisfied, the method 1500 proceeds to block 1560 in some embodiments, or the method proceeds to block 1570 in some other embodiments.

At block 1560, the processor 240 determines to execute the EMI risk mitigation function, then the method 1500 proceeds to block 1570. At block 1570, the processor 240 executes or performs the EMI risk mitigation function. The processor 240 performs the EMI risk mitigation function by adjusting a radio frequency (RF) transmit power, or outputting an alert that warns a user of the electronic device about a pacemaker in relation to the detected EMI risk. In some embodiments, processor 240 performs the EMI risk mitigation function by both adjusting a radio frequency (RF) transmit power and outputting an alert that warns a user of the electronic device about a pacemaker in relation to the detected EMI risk.

Although FIG. 15 illustrates an example method 1500 for warning a wearer of a pacemaker against potential electromagnetic interference by a radar-aided mobile device, various changes may be made to FIG. 15. For example, while shown as a series of steps, various steps in FIG. 15 could overlap, occur in parallel, occur in a different order, or occur any number of times. As a particular multiple-stage example, at block 1510, the processor 240 can obtain the sensor data 602 from the sensor further by obtaining first sensor data from a primary sensor, wherein the sensor includes a primary sensor that includes at least one among a radar 400 and an IMU of the electronic device 200. Further at block 1540 of this multiple-stage example, the processor 240, in response to a determination that the first sensor data does not satisfy the condition for executing the EMI risk mitigation function, determines to: (i) when the primary sensor includes both the radar and the IMU, operate the electronic device without executing the EMI risk mitigation function; and (ii) when the primary sensor does not include both the radar and the IMU, obtain second sensor data from a secondary sensor. The secondary sensor is different from the primary sensor. The secondary sensor is the IMU when the primary sensor is the radar, and the secondary sensor is the radar when the primary sensor is the IMU.

The multiple-stage example is further described in reference to FIGS. 11 and 14.

The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the figures illustrate different examples of user equipment, various changes may be made to the figures. For example, the user equipment can include any number of each component in any suitable arrangement. In general, the figures do not limit the scope of this disclosure to any particular configuration(s). Moreover, while figures illustrate operational environments in which various user equipment features disclosed in this patent document can be used, these features can be used in any other suitable system.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claims scope. The scope of patented subject matter is defined by the claims.

What is claimed is:

1. A method comprising:
obtaining sensor data from a sensor of an electronic device;
detecting electromagnetic interference (EMI) risk based on the sensor data obtained;
determining whether a condition for executing an EMI risk mitigation function is satisfied, based on the detected EMI risk; and
in response to determining that the condition for executing the EMI risk mitigation function is satisfied, performing the EMI risk mitigation function.

2. The method of claim 1, wherein performing the EMI risk mitigation function comprises at least one of:
adjusting a radio frequency (RF) transmit power; and
outputting an alert that warns a user of the electronic device about a pacemaker in relation to the detected EMI risk.

3. The method of claim 1, further comprising:
in response to determining that the condition for executing the EMI risk mitigation function is not satisfied, operating the electronic device without executing the EMI risk mitigation function.

4. The method of claim 1, wherein:
detecting the EMI risk further comprises estimating a distance to a heart of a user of the electronic device; and
determining whether the condition for executing an EMI risk mitigation function is satisfied further comprises determining whether the distance to the heart satisfies a proximity condition.

5. The method of claim 1, wherein:
obtaining the sensor data from the sensor of the electronic device further comprises obtaining radar data into a sliding input data window that includes recent radar frames from the radar data; and
the method further comprises:
filtering the data window to pass through a subset of the radar data that matches a typical frequency range of a heartbeat; and
detecting that a heartbeat is present based on a determination that an energy of the subset of the radar data satisfies a threshold energy condition.

6. The method of claim 5, further comprising:
receiving heart rate estimation information from an external electronic device worn by a user of the electronic device; and
determining the typical frequency range of a heartbeat based on the heart rate estimate received.

7. The method of claim 1, wherein:
obtaining the sensor data from the sensor of the electronic device further comprises obtaining inertial measurement unit (IMU) data corresponding to a sliding input data window; and
the method further comprises:
classifying the IMU data as one from among a set of EMI risk classes, the set of EMI risk classes including:
a first class indicating that a location of the electronic device is IN a location of interest, and
a second class indicating that the location of the electronic device is OUT of the location of interest;
determining that the condition for executing the EMI risk mitigation function is satisfied, based on the IMU data classified as the first class; and
determining that the condition for executing the EMI risk mitigation function is not satisfied, based on the IMU data classified as the second class.

8. The method of claim 7, wherein:
the set of EMI risk classes includes the first class, the second class, and a third class indicating that the location of the electronic device is UNDETERMINED relative to the location of interest; and
detecting the EMI risk further comprises activating a radar of the electronic device, based on the IMU data classified as the third class.

9. The method of claim 1, wherein:
obtaining the sensor data from the sensor further comprises obtaining first sensor data from a primary sensor, wherein the sensor includes a primary sensor that includes at least one among a radar and an inertial measurement unit (IMU) of the electronic device; and
the method further comprises, in response to a determination that the first sensor data does not satisfy the condition for executing the EMI risk mitigation function, determining to:
when the primary sensor includes both the radar and the IMU, operate the electronic device without executing the EMI risk mitigation function; and
when the primary sensor does not include both the radar and the IMU, obtain second sensor data from a secondary sensor, the secondary sensor being different from the primary sensor and being the IMU when the primary sensor is the radar, and the secondary sensor being the radar when the primary sensor is the IMU.

10. The method of claim 1, further comprising detecting the EMI risk based on identifying a setting in which pacemaker compatibility is activated.

11. An electronic device comprising:
a radio frequency (RF) transceiver; and
a processor configured to:
obtain sensor data from a sensor of the electronic device;
detect electromagnetic interference (EMI) risk based on the sensor data obtained;
determine whether a condition for executing an EMI risk mitigation function is satisfied, based on the detected EMI risk; and
in response to determining that the condition for executing the EMI risk mitigation function is satisfied, performing the EMI risk mitigation function.

12. The electronic device of claim 11, wherein to perform the EMI risk mitigation function, the processor is further configured to at least one of:
adjust an RF transmit power of the RF transceiver; and
output an alert that warns a user of the electronic device about a pacemaker in relation to the detected EMI risk.

13. The electronic device of claim 11, wherein the processor is further configured to:
in response to determining that the condition for executing the EMI risk mitigation function is not satisfied, operate the electronic device without executing the EMI risk mitigation function.

14. The electronic device of claim 11, wherein:
to detecting the EMI risk, the processor is further configured to estimate a distance to a heart of a user of the electronic device; and
to determine whether the condition for executing an EMI risk mitigation function is satisfied, the processor is further configured to determine whether the distance to the heart satisfies a proximity condition.

15. The electronic device of claim 11, wherein:
to obtain the sensor data from the sensor of the electronic device, the processor is further configured to obtain radar data into a sliding input data window that includes recent radar frames from the radar data; and
the processor is further configured to:
filter the data window to pass through a subset of the radar data that matches a typical frequency range of a heartbeat; and
detect that a heartbeat is present based on a determination that an energy of the subset of the radar data satisfies a threshold energy condition.

16. The electronic device of claim 15, wherein the processor is further configured to:
receive heart rate estimation information from an external electronic device worn by a user of the electronic device; and
determine the typical frequency range of a heartbeat based on the heart rate estimate received.

17. The electronic device of claim 11, wherein:
to obtain the sensor data from the sensor of the electronic device, the processor is further configured to obtain inertial measurement unit (IMU) data corresponding to a sliding input data window; and
the processor is further configured to:
classify the IMU data as one from among a set of EMI risk classes, the set of EMI risk classes including:
a first class indicating that a location of the electronic device is IN a location of interest, and
a second class indicating that the location of the electronic device is OUT of the location of interest;
determine that the condition for executing the EMI risk mitigation function is satisfied, based on the IMU data classified as the first class; and
determine that the condition for executing the EMI risk mitigation function is not satisfied, based on the IMU data classified as the second class.

18. The electronic device of claim 17, wherein:
the set of EMI risk classes includes the first class, the second class, and a third class indicating that the location of the electronic device is UNDETERMINED relative to the location of interest; and
to detect the EMI risk, the processor is further configured to activate a radar of the electronic device, based on the IMU data classified as the third class.

19. The electronic device of claim 11, wherein:
to obtain the sensor data from the sensor, the processor is further configured to obtain first sensor data from a primary sensor, wherein the sensor includes a primary sensor that includes at least one among a radar and an inertial measurement unit (IMU) of the electronic device; and
the processor is further configured to, in response to a determination that the first sensor data does not satisfy the condition for executing the EMI risk mitigation function, determine to:
when the primary sensor includes both the radar and the IMU, operate the electronic device without executing the EMI risk mitigation function; and
when the primary sensor does not include both the radar and the IMU, obtain second sensor data from a secondary sensor, the secondary sensor being different from the primary sensor and being the IMU when the primary sensor is the radar, and the secondary sensor being the radar when the primary sensor is the IMU.

20. The electronic device of claim 11, wherein the processor is further configured to detect the EMI risk based on identifying a setting in which pacemaker compatibility is activated.
